(19) United States
(12) Patent Application Publication
(10) Pub. No.: US 2026/0048024 A1
(43) Pub. Date: Feb. 19, 2026

(54) ACID-RESPONSIVE POLYMER ADDITIVES INCREASE RNA TRANSFECTION FROM LIPID NANOPARTICLES

(71) Applicant: The Governing Council of the University of Toronto, Toronto (CA)

(72) Inventors: Timothy Hue-Fung CHEUNG, Toronto (CA); Molly Sandra SHOICHET, Toronto (CA)

(21) Appl. No.: 19/281,389

(22) Filed: Jul. 25, 2025

Related U.S. Application Data
(60) Provisional application No. 63/683,371, filed on Aug. 15, 2024.

(51) Int. Cl. A61K 9/51; A61K 31/7105; A61K 31/713; C08G 63/08; C08G 63/78; C08G 81/00

(52) U.S. Cl. CPC A61K 9/5192; A61K 9/5153; A61K 31/7105; A61K 31/713; C08G 63/08; C08G 63/78; C08G 81/00

(57) ABSTRACT

Lipid nanoparticles (LNPs) are widely used for RNA delivery but are limited by inefficient RNA release following endosomal escape. Disclosed herein are hybrid polymer-lipid nanoparticles (PLNPs) incorporating acid-responsive poly(lactic acid)-block-poly(carboxybetaine) zwitterionic polymers to enhance RNA delivery efficiency. The polymers are cationic at physiological pH to enable RNA complexation but become neutral at endosomal pH, reducing RNA binding affinity and promoting release. These polymers were integrated into clinically approved LNP formulations to form PLNPs. The resulting PLNPs showed up to a 5.4-fold decrease in siRNA IC50 values and a 4-fold increase in mRNA transfection across multiple cell lines. Enhanced cytosolic RNA levels were confirmed via confocal microscopy, with uptake and endosomal escape comparable to standard LNPs. The improvement in transfection efficiency was lost when acid-inert polymers were used, confirming the role of the acid-responsive polymers. This approach provides a versatile platform to improve RNA delivery from existing LNP systems.

### CROSS REFERENCE TO RELATED APPLICATIONS

The present application claims priority to U.S. Provisional Patent Application No. 63/683,371 filed Aug. 15, 2024, the contents of which are hereby incorporated by reference in their entirety.

### FIELD

The present disclosure relates to acid-sensitive polymers to increase RNA dissociation from lipid nanoparticles after endocytosis into cells. More particularly, herein there has been synthesized three acid-responsive poly(lactic acid)-block-poly(carboxybetaine) zwitterionic derivatives that were cationic and complexed with RNA at pH 7.4, but were neutral following cleavage at endosomal pH, thereby having lower affinity to RNA.

### BACKGROUND

RNA therapeutics are an emerging class of biologics with enormous potential. In 2018, the FDA approved the first siRNA therapeutic, Onpattro, and only a few years later, Comirnaty and Spikevax were approved as the first mRNA vaccines. While the mechanisms and potential of RNA therapeutics are now well-known, the intracellular delivery of these macromolecules remains suboptimal. Most FDA-approved RNA therapeutics currently use lipid nanoparticles (LNPs) for intracellular delivery;^{[1,2]} however, LNPs are notoriously inefficient. Several groups have demonstrated that only a small percentage (<5%) of RNA endocytosed by a cell escapes from the endosome and is available to the cellular machinery.^{[3-5]} Thus, while LNPs are the most clinically-advanced RNA delivery vehicle, their low delivery efficiency necessitates a larger dose than necessary, and increases adverse effects such as immunogenicity^{[6.7]} and toxicity with cationic or ionizable lipids.^{[8]} One potential solution to increasing RNA delivery efficiency is improving endosomal escape; however, engineering methods to induce endosomal escape are often correlated with greater cytotoxicity, which limits the potential of these solutions.^{[9,10]} Thus, there is a need to develop other methods to increase RNA delivery efficiency without causing toxicity.

### SUMMARY

The inventors considered whether RNA dissociation from the LNPs could be manipulated after cellular internalisation to make more RNA available to the cellular machinery in the cytosol. Several studies have demonstrated that only a small percentage of RNA within a given endosome reaches the cytosol,^{[3-5]} suggesting that a significant amount of RNA remains trapped in the LNP even after endosome disruption and are therefore degraded in the lysosome.^{[11,12]} Several groups have developed nanocarriers that dissociate from RNA to address this problem. For example, Greco et al. used polymeric nanocarriers that disassemble under UV light to increase siRNA silencing^{[13]} while other groups have designed acid-responsive self-immolating polymers which dissociate into monomers under acidic conditions for mRNA delivery.^{[14-17]} Such polymeric approaches come with their own challenges. For example, polymeric nanocarriers typically require high positive charge to enter cells, but this high charge can also disrupt cell membranes and induce cytotoxicity.^{[18,19]} Additionally, most polyplexes fuse poorly with the endosomal membrane and therefore have poor endosomal escape, meaning that even though the RNA is released from its carrier, it is still mostly trapped within the endosome.^{[20]} To overcome the limitations of polymers, others have proposed small-molecule solutions for RNA dissociation, such as lipids sensitive to reactive oxygen species (ROS)^{[21]} and acid-responsive surfactants. However, using these novel molecules would preclude the use of many new lipids being discovered with enhanced efficiency over current lipids.^{[23-25]} The inventors therefore sought to design a generalizable strategy to improve RNA release from multiple LNPs.

The inventors hypothesized that an acid-responsive RNA-releasing polymer could be used in LNP formulations to achieve greater RNA release and transfection efficiency. Polymers have been formulated with LNPs previously for various applications. For example, poly(lactide-co-glycolide) (PLGA) has been incorporated into LNP cores for greater serum stability^{[26-28]} while poly(β-aminoester) (PBAE) nanoparticles (NPs) have been coated in lipids to increase both uptake into target cells and biocompatibility.^{[29-31]} Hybrid nanoparticles of cationic peptides and lipids have also been shown to increase RNA delivery over peptide or lipid nanoparticles alone.^{[32]} While these hybrid nanoparticles showed promising results, RNA dissociation from its carrier was not a primary consideration. Thus, the inventors hypothesized that RNA-releasing, acid-responsive polymers incorporated into LNPs would increase RNA delivery by facilitating RNA dissociation from its nanocarrier.

The present disclosure provides acid-sensitive polymers to increase RNA dissociation from lipid nanoparticles after endocytosis into cells. Specifically, the inventors have synthesized three acid-responsive poly(lactic acid)-block-poly(carboxybetaine) zwitterionic derivatives that were cationic and complexed with RNA at pH 7.4, but were neutral following cleavage at endosomal pH, thereby having lower affinity to RNA. This charge transition was achieved by protecting the pendant carboxylate groups with hemiacetal esters. These polymers have not been reported in the literature and are notable for their unusual positive to neutral transition at acidic pH.

The utility of these polymers has been demonstrated by formulating them into the clinically approved Onpattro, Moderna, or Pfizer lipid nanoparticle (LNP) formulations to produce hybrid polymer-lipid nanoparticles (PLNPs). The PLNPs decreased the IC50 values of multiple siRNAs up to 5.4-fold compared to parent LNPs in several cell lines. Moreover, mRNA transfection increased up to 4-fold. Mechanistic studies confirmed that acid-responsive cleavage of the hemiacetal pendant groups on the polymer was necessary to achieve this effect. Thus, the inventors have demonstrated that this polymer is a useful additive to improve LNP delivery efficiency and has diverse applications.

Thus, the present disclosure provides for the synthesis of novel amphiphilic poly(lactic acid)-block-poly(carboxybetaine) (PLA-b-PCB) zwitterionic polymers that are converted from positive to neutral in acidic conditions. Specifically, the inventors modified PLA-b-PCB polymers^{[33]} with acid-responsive hemiacetal ester pendant groups (PLA-b-PCB-X). These groups are most often used as acid-labile protecting groups in solid-phase peptide synthesis and were chosen for acid-responsive RNA release because they are highly labile in mildly acidic conditions. The PLA-b-PCB-X polymers were chosen because their amphiphilicity would allow mixing into LNPs thereby creating novel, hybrid polymer-lipid nanoparticles (PLNPs). Moreover, both PLA and PCB are biocompatible and readily excreted from the body, thus reducing the risk of cytotoxicity after RNA delivery.^{[33,35,36]}

The inventors demonstrate that these PLA-b-PCB-X polymers can be used as additives in existing LNP formulations to produce stable PLNPs encapsulating either siRNA or mRNA with sizes less than 200 nm, which is preferred for endocytosis.^{[37]} The PLNP formulations were based on the clinically approved Onpattro formulation, which consists of DLin-MC3-DMA ionizable lipid (MC3), Distearoylphosphatidylcholine (DSPC), cholesterol and 1,2-dimyristoyl-rac-glycero-3-methoxypolyethylene glycol-2000 (DMG-PEG₂₀₀₀ or DMG-PEG_{2K}) in mass ratios of approximately 50:19:25:6,^{[38]} and were formulated using microfluidic mixing, which is the gold standard method for siRNA LNP formulation.^{[39,40]} The inventors examined the effect of our polymers on siRNA and mRNA delivery using multiple LNP formulations including the Onpattro, Pfizer and Moderna formulations in multiple cell lines and also compared uptake and endosomal escape of our PLNPs versus conventional LNPs and determined how acid-responsive RNA release affected cytosolic RNA concentration. The inventors demonstrate the importance of RNA release from its nanocarrier and propose inclusion of our PLA-b-PCB-X polymers in LNPs as a simple and versatile method to improve RNA delivery efficiency (FIG. 1).

There is provided a pH-sensitive polymer, a method for the preparation thereof, a use of a pH-sensitive polymer for preparation of lipid nanoparticles containing nucleic acids, and a method of delivering RNA to cytosol of cells, using a pH-sensitive polymer. According to an aspect, there is provided a pH-sensitive polymer. The pH-sensitive polymer comprises a poly(lactic acid)-block-poly(carboxybetaine) derivative that is cationic and complexed with a nucleic acid at pH 7.4 and irreversibly converted to neutral and having a lower affinity to the nucleic acid at endosomal pH.

In a particular case, the poly(lactic acid)-block-poly(carboxybetaine) derivative is selected from the group consisting of amphiphilic, cationic block co-polymer PLA-b-PCB-THP; amphiphilic, cationic block co-polymer PLA-b-PCB-EOE; and amphiphilic, cationic block co-polymer PLA-b-PCB-MOM.

In another case, the poly(lactic acid)-block-poly(carboxybetaine) derivative is amphiphilic, cationic block co-polymer PLA-b-PCB-THP.

In yet another case, the poly(lactic acid)-block-poly(carboxybetaine) derivative is amphiphilic, cationic block co-polymer PLA-b-PCB-EOE.

In yet another case, the poly(lactic acid)-block-poly(carboxybetaine) derivative is amphiphilic, cationic block co-polymer PLA-b-PCB-MOM.

Accordingly, in an aspect, there is provided a method of synthesizing a pH-sensitive cationic block co-polymer comprising a poly(lactic acid)-block-poly(carboxybetaine) derivative of formula PLA-b-PCB-X. The method comprises the steps of synthesizing an intermediate of formula X-Br; and carrying out alkylation of PLA-b-PDMAPMA by X-Br to produce amphiphilic, cationic block co-polymer PLA-b-PCB-X, wherein X-Br is tetrahydro-2H-pyran-2-yl 2-bromoacetate (THP-Br), 1-ethoxyethyl 2-bromoacetate (EOE-Br) or methoxymethyl 2-bromoacetate (MOM-Br) and the block co-polymer PLA-b-PCB-X is PLA-b-PCB-THP, PLA-b-PCB-EOE or PLA-b-PCB-MOM.

In a particular case of this method, PLA-b-PCB-X is PLA-b-PCB-THP and X-Br is tetrahydro-2H-pyran-2-yl 2-bromoacetate (THP-Br).

In another case of this method, PLA-b-PCB-X is PLA-b-PCB-EOE and X-Br is 1-ethoxyethyl 2-bromoacetate (EOE-Br).

In yet another case of this method, PLA-b-PCB-X is PLA-b-PCB-MOM and X-Br is methoxymethyl 2-bromoacetate (MOM-Br).

In another case, poly(lactic acid)-block-poly(carboxybetaine) derivative are used for preparation of lipid nanoparticles containing nucleic acids.

In yet another case, the nucleic acids are RNA.

In yet another case, the RNA is mRNA or siRNA.

Accordingly, in an aspect, there is provided a method of delivering RNA to cytosol of cells, using a pH-sensitive polymer. The method comprises (a) preparing a formulation of lipid nanoparticles with RNA at pH 7.4 in the presence of the pH-sensitive polymer, and (b) transfecting cells with the formulation obtained from step (a).

In another case of this method, the RNA is mRNA or siRNA.

Accordingly, in an aspect, there is provided a pH-sensitive polymer. The pH-sensitive polymer comprises a poly(lactic acid)-block-poly(carboxybetaine) derivative having pendant carboxylate groups protected with hemiacetal esters.

In a particular case, the poly(lactic acid)-block-poly(carboxybetaine) derivative is selected from the group consisting of amphiphilic, cationic block co-polymer PLA-b-PCB-THP; amphiphilic, cationic block co-polymer PLA-b-PCB-EOE; and amphiphilic, cationic block co-polymer PLA-b-PCB-MOM.

In another case, the poly(lactic acid)-block-poly(carboxybetaine) derivative is used for the preparation of lipid nanoparticles containing nucleic acids.

In yet another case, the nucleic acids are RNA.

Accordingly, in an aspect, there is provided a method of delivering RNA to cytosol of cells, using a pH-sensitive polymer. The method comprises (a) preparing a formulation of lipid nanoparticles with RNA at pH 7.4 in the presence of the pH-sensitive polymer, and (b) transfecting cells with the formulation obtained from step (a).

In a particular case, the RNA is mRNA or siRNA.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1: Concept of PLNP endocytosis and endosomal escape of RNA from PLNPs into the cytosol of cells. Created with Biorender.com.

FIG. 2A-2E: Synthesis and characterization of acid responsive polymers PLA-b-PCB-tetrahydropyranyl (THP), -ethoxyethyl (EOE) and -methoxymethyl (MOM). FIG. 2A: Synthesis of PLA-b-PCB-X derivatives-THP, -EOE, and -MOM from poly(lactic acid)-block-poly(N-[3-(dimethylamino)propyl]methacrylamide) (PLA-b-PDMAPMA). FIG. 2B: Zeta potential of self-assembled nanoparticles of PLA-b-PCB-THP, -EOE and -MOM polymers immediately after formulation (0 h) and 24 h at 37° C. at either pH 7.4 or pH 4.5 (n=3 independent formulations). FIG. 2C: Proposed mechanism for acid-responsive hydrolysis and charge neutralization of pendant groups of PLA-b-PCB-X polymers. FIG. 2D: Gel retardation assay performed with polymers incubated at 37° C. for 24 h at either pH 7.4 or pH 4.5. siRNA was incubated with polymers, then free RNA was imaged in a tris-borate-EDTA (TBE) gel after staining with SYBR Safe DNA stain. FIG. 2E Quantification of gel retardation assay results, n=3 independent experiments. For FIG. 2B and FIG. 2D, data are presented as mean±standard deviation (SD), and statistics with a two-way ANOVA with Tukey's post-hoc test, *p<0.05, **p<0.01.

FIG. 3A-3F: Formulation and characterization of hybrid PLA-b-PCB-X polymer/MC3 PLNPs. FIG. 3A: Z-average size and PDI of PLNPs as measured by DLS. Data presented as mean±SD of 20 measurements. FIG. 3B: RNA encapsulation efficiency of PLNPs as measured by RiboGreen assay. Data presented as mean±SD of 3 technical replicates. Representative cryo-TEM images of 50/0 PLA-b-PCB-MOM/MC3 (FIG. 3C), 25/25 PLA-b-PCB-MOM/MC3 (FIG. 3D) and \ 0/50 PLA-b-PCB-MOM/MC3 PLNPs (FIG. 3E). Scale bar=200 nm. FIG. 3F: Proposed structure of mixed PLA-b-PCB-X/MC3 PLNPs.

FIG. 4A-4G: PLNPs enhance siRNA-mediated gene knockdown. FIG. 4A: Dose-response curves based on luminescence of 25/25 and 12.5/37.5 PLA-b-PCB-X/MC3 PLNPs encapsulating siFLuc in SK-OV-3-FLuc reporter cells (n=4-6 biological replicates). FIG. 4B: Quantification of IC₅₀ values from (FIG. 4A) (n=4-6 biological replicates, p values calculated by one-way ANOVA with Tukey's post-hoc test, *p<0.05, **p<0.01). FIG. 4C: Dose-response curves based on luminescence of 25/25 and 12.5/37.5 PLA-b-PCB-X/ALC-0315 PLNPs encapsulating siFLuc in SK-OV-3-FLuc cells (n=4 biological replicates). FIG. 4D: Dose-response curves based on luminescence of 25/25 and 12.5/37.5 PLA-b-PCB-X/SM-102 PLNPs encapsulating siFLuc in SK-OV-3-FLuc cells (n=4 biological replicates). FIG. 4E: Quantification of IC₅₀ values from FIG. 4C and FIG. 4D (p values calculated by one-way ANOVA with Tukey's post-hoc test, *p<0.05, ***p<0.001). FIG. 4F: Metabolic activity of CCNE1-overexpressing T-47D cells after dosing for 24 h with specified treatments as measured by the PrestoBlue assay (n=3 biological replicates). FIG. 4G: Dose-response curves of CCNE1 knockdown with PLNPs encapsulating siCCNE1 in CCNE1-overexpressing T-47D cells (n=3 biological replicates, p values calculated by two-way ANOVA with Tukey's post-hoc test. Data are presented as mean±SD, *p<0.05, **p<0.01, ***p<0.001, ****p<0.0001 vs. siFLuc (PLNP), #p<0.05 vs. siCCNE1 (SM)).

FIG. 5A-5F: Acid-responsive hydrolytic group increases siRNA bioavailability in the cytosol. FIG. 5A: Chemical structures of pendant groups used for experiments. FIG. 5B Dose-response curves of luminescence vs. siFLuc delivered in PLNPs to SK-OV-3-FLuc cells (n=4-6 biological replicates, data for 50 MC3 and 12.5/37.5 PLA-b-PCB-MOM/MC3 are reproduced from FIG. 4A for comparison). FIG. 5C: IC₅₀ values for data in FIG. 5B. Data for 50 MC3 and 12.5/37.5 PLA-b-PCB-MOM/MC3 are reproduced from FIG. 4B for comparison. FIG. 5D: Representative images of Cy3-siRNA distribution in SK-OV-3 cells. Scale bar=50 μm. FIG. 5E: Area in pixels of Cy3 signal per cell (n=8 biological replicates). FIG. 5F: Total Cy3 pixel intensity per cell, as calculated by raw integrated density function in ImageJ (n=8 biological replicates, p values calculated using one-way ANOVA with Tukey's post-hoc test). Data are presented as mean±SD, *p<0.05, ***p<0.001.

FIG. 6A-6D: Uptake and endosomal escape of PLNPs in SK-OV-3-mChG9 cells. FIG. 6A: Representative images of uptake and endosomal escape in SK-OV-3 cells. Scale bar=50 μm. FIG. 6B: Number of NP puncta per cell as visualized using the DiD channel (n=4 biological replicates, p values calculated using one-way ANOVA with Tukey's post-hoc test). FIG. 6C: Number of disrupted endosomes per cell as visualized by Gal9-mCherry foci (n=4 biological replicates, p values calculated using one-way ANOVA with Tukey's post-hoc test). FIG. 6D: NP puncta per cell after pre-treatment with endocytosis inhibitors (n=3 biological replicates, p values calculated using two-way ANOVA with Sidak's post-hoc test). Data are presented as mean±SD, ns=not significant, *p<0.05, ***p<0.001.

FIG. 7A-7E: Formulation and delivery of PLNPs encapsulating FLuc mRNA. FIG. 7A: Size and PDI of 12.5/37.5 PLA-b-PCB-X/MC3 PLNPs encapsulating FLuc mRNA as measured by DLS (n=3 technical replicates). FIG. 7B: Zeta potential of PLNPs (n=3 technical replicates). FIG. 7C: Encapsulation efficiency of PLNPs as measured by RiboGreen assay (n=4 technical replicates). FIG. 7D: Metabolic activity of SK-OV-3 cells after dosing with treatments. FIG. 7E: Dose-response curves of luminescence per microgram total protein content vs. FLuc mRNA in SK-OV-3 cells (n=4 biological replicates, data are presented as mean±SD, ***p<0.001, calculated by two-way ANOVA with Tukey's post-hoc test).

FIG. 8A-8B: Representative ¹H NMR spectra of PLA-b-PDMAPMA (FIG. 8A) and PLA-b-PCB-MOM (FIG. 8B). FIG. 8A was taken in DMF-d₇ on a 500 MHz spectrometer. FIG. 8B was taken in 1:1 CD₃CN:D₂O on a 400 MHz spectrometer.

FIG. 9: Representative image of full gel from gel retardation assays. NPs were incubated with 0.5 μg siFLuc after incubation in various buffers as described in the Experimental Section. Free RNA was then separated by gel electrophoresis. RNA adsorption was calculated by determination of free RNA by comparison of band intensity against the calibration curve on the same gel.

FIG. 10A-10B: Particle size and dispersity of polymer-only micelles incubated with RNA. FIG. 10A: Z-average size of nanoparticles (NPs) alone, NPs with RNA immediately after mixing, and NPs with RNA 3 d after mixing as measured by DLS. FIG. 10B: PDI of samples described in FIG. 10A. Synthesis of PLA-b-PCB-CONHEt and PLA-b-PCB-COOtBu polymers are described in a previous work.^{[1]} Data are presented as mean±SD, n=3 technical replicates.

FIG. 11: Z-average size and PDI of PLNPs with varying amounts of PLA-b-PCB-COOEt polymer and DSPC. The amounts of cholesterol and DMG-PEG₂ₖ were kept constant at 25% and 6%, respectively. Data are presented as mean±SD, n=3 technical replicates.

FIG. 12: Z-average size and PDI of PLNPs with varying amounts of DSPC and cholesterol. The amounts of PLA-b-PCB-COOEt and DMG-PEG₂ₖ were kept constant at 50% and 6%, respectively. Data are presented as mean±SD, n=3 technical replicates.

FIG. 13A-13B: FIG. 13A: Z-average size and FIG. 13B: zeta potential of PLNPs with varying amounts of DSPC and DMG-PEG₂ₖ. The amounts of PLA-b-PCB-COOEt and cholesterol were kept constant at 50 and 25 wt %, respectively. Data are presented as mean±SD, n=3 technical replicates.

FIG. 14A-14D: FIG. 14A: Z-average size, FIG. 14B: RNA encapsulation efficiency, and FIG. 14C: zeta potential of PLNPs with either DSPC, 1,2-dioleoyl-sn-glycero-3-phosphocholine (DOPC), or 1,2-dioleoyl-sn-glycero-3-phosphoethanolamine (DOPE) as the structural lipid and FIG. 14D composition of nanoparticles characterized in FIG. 14A-C. Data in FIG. 14A and FIG. 14C are presented as mean±SD, n=3 technical replicates.

FIG. 15: Additional cryo-TEM images of 50/0, 25/25, and 0/50 PLA-b-PCB-MOM/MC3 PLNPs. Scale bar=50 nm

FIG. 16: Full synthetic pathway for DSPE-PCB polymers.

FIG. 17: ¹H NMR spectrum of DSPE-CTA in CDCl₃.

FIG. 18: ¹H NMR spectrum of DSPE-PDMAPMA in DMSO-d₆.

FIG. 19: Representative ¹H NMR spectrum of DSPE-THP polymer in CD₃OD.

FIG. 20A-20B: Size and RNA encapsulation of DSPE-PCB-MOM PLNPs. FIG. 20A: Z-average size of PLNPs measured by DLS. FIG. 20B: RNA encapsulation efficiency of PLNPs measured by RiboGreen assay. Data are presented as mean±SD, n=3 formulations.

FIG. 21: Cryo-TEM images of 50/0, 25/25, and 12.5/37.5 DSPE-PCB-MOM/MC3 PLNPs. Scale bar=200 nm.

FIG. 22A-22C: Knockdown of FLuc in SK-OV-3-FLuc cells by PLNPs with varying ratios of PLA-b-PCB-THP/MC3 (FIG. 22A), PLA-b-PCB-EOE/MC3 (FIG. 22B), and PLA-b-PCB-MOM/MC3 (FIG. 22C), as measured by luminescence in a Steady-Glo assay. Data are presented as mean±SD, n=3 technical replicates.

FIG. 23A-23B: Metabolic activity of SK-OV-3-FLuc cells after treatment with PLNPs. FIG. 23A: PrestoBlue assay was performed after treatment with PLA-b-PCB-X/MC3 PLNPs. Data are presented as mean±SD, n=4-6 biological replicates. *p<0.05, calculated by two-way ANOVA with Tukey's post-hoc. FIG. 23B: Cells were treated with PLA-b-PCB-MOM/ALC or PLA-b-PCB-MOM/SM PLNPs, then metabolic assay was measured by PrestoBlue assay. Data are presented as mean±SD, n=4 biological replicates.

FIG. 24A-24C: Comparison of uptake and endosomal escape in SK-OV-3-mChG9 cells using PLA-b-PCB polymers and DSPE-PCB polymers. FIG. 24A: NP puncta and FIG. 24B: Gal9 foci in SK-OV-3-mChG9 cells after treatment with 10 nM siRNA in various PLNPs for 3 h. Data are presented as mean±SD, n=4 biological replicates. FIG. 24C: Endosomal escape efficiency can be compared by plotting Gal9 foci per cell and NP puncta per cell and taking the slope of the line of best fit. Each point represents a single technical replicate (i.e. single well).

FIG. 25A-25B: PLA-b-PCB-MOM/MC3 and DSPE-PCB-MOM/MC3 PLNPs are taken up by fulvestrant-resistant T-47D (FIG. 25A) and HepG2 cells (FIG. 25B). 10 nM siRNA in various DiD-labelled PLNPs were incubated with cells 3 h, then cells were imaged by confocal microscopy. Data are presented as mean±SD, n=3 biological replicates.

FIG. 26: Hydroxydynasore inhibits PLNP uptake into fulvestrant-resistant T-47D cells. Cells were pre-treated with endocytosis inhibitors for 30 min, then 10 nM siRNA in PLNPs were added. After 3 h, PLNPs were removed and cells were fixed and imaged. Data are presented as mean±SD, n=3 biological replicates.

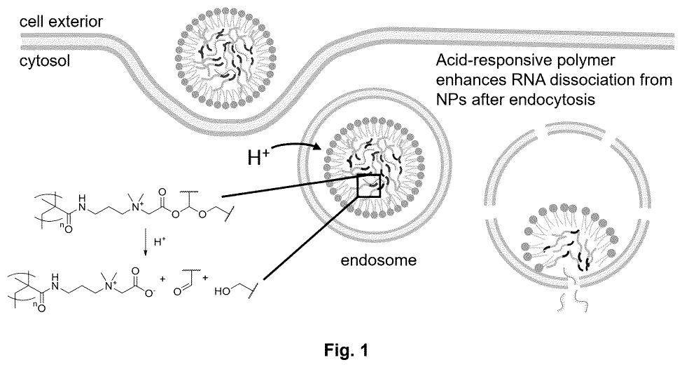

### DETAILED DESCRIPTION

A detailed description is provided below to facilitate a thorough understanding of the disclosed embodiments and connections thereof. The description is not limited to any particular example included herein.

Various embodiments and aspects of the disclosure will be described with reference to the details discussed below. The following description and drawings are illustrative of the disclosure and are not to be construed as limiting the disclosure. Numerous specific details are described to provide a thorough understanding of various embodiments of the present disclosure. The Figures are not to scale. Further, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately”, when used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

### Experimental Section

### Materials

The following were purchased from Sigma-Aldrich (St. Louis, USA) and used as received unless otherwise noted: 3,6-dimethyl-1,4-dioxane-2,5-dione (D,L-lactide), tin(II) 2-ethylhexanoate, 4-cyano-4-[(dodecylsulfanylthiocarbonyl)sulfanyl]pentanol (CTA-OH), N-[3-(dimethylamino)propyl]methacrylamide (DMAPMA), 2,2′-azobis(2-methylpropionitrile) (AIBN), bromoacetic acid, 3,4-dihydro-2H-pyran, cholesterol, 2,2,2-trifluoroethanol, deuterium oxide, methanol-d₄. Ethyl vinyl ether and bromomethyl methyl ether were purchased from TCI Chemicals (Tokyo, Japan). Genistein, nystatin, chlorpromazine, amiloride, hydroxydynasore and DLin-MC3-DMA were purchased from MedChemExpress (Monmouth Junction, USA). Toluene, 1,4-dioxane, dimethylformamide (DMF), dichloromethane (DCM), acetonitrile-d₃ and methanol were purchased from Caledon Laboratories (Halton Hills, Canada). DSPC and DMG-PEG₂ₖ were purchased from Avanti Polar Lipids. Ethanol was purchased from Commercial Alcohols Inc. (Brampton, Canada). Chloroform-d and DMSO-d₆ were purchased from Cambridge Isotope Laboratories (St. Laurent, Canada). EZ Cap Firefly Luciferase mRNA was purchased from ApexBio (Houston, USA). siRNA and DNA primers were synthesized by Integrated DNA Technologies (Coralville, USA).

siRNA sequences

| Gene | Sequence |
|---|---|
| siFLuc | 5′-mGmGUmUCmCUGGAAmCAmAUmUGmCUUUUAmCdA-3′ |
| | 5′-UGMUAAAAGmCAmAUmUGUUCCAGGAmACmCmAmG-3′ |
| siCCNE1 | 5′-mGmCUmUCmGGCCUUmGUmAUmCAmUUUCUCmGT-3′ |
| | 5′-ACmGAGAAAmUGmAUmACAAGGCCGAmAGmCmUmG-3′ |

m = 2′-O-methylated RNA base. d = DNA base.
qPCR primers

| Gene | Sequence |
|---|---|
| CCNE1 | Forward: 5′-TGTGTCCTGGATGTTGACTGCC-3′ |
| | Reverse: 5′-CTCTATGTCGCACCACTGATACC-3′ |
| ACTB | Forward: |
| | 5′-CCAACCGCGAGAAGATGACCCAGATCATGT-3′ |
| | Reverse: |
| | 5′-GTGAGGATCTTCATGAGGTAGTCAGTCAGG-3′ |

Silencer™ Cy™3-labeled Negative Control No. 1 siRNA (Cy3-siRNA), Quant-iT RiboGreen RNA reagent, SYBR Safe DNA Gel Stain, RPMI 1640 and DMEM were purchased from Thermo Fisher (Waltham, USA). SK-OV-3, SK-OV-3-FLuc, and HepG2 cells were purchased from ATCC (Manassas, USA). Palbociclib-resistant T-47D cells were a generous gift from Prof. David Cescon (University Health Network, Canada) and were generated as previously described.^{[53]} Fetal bovine serum and penicillin-streptomycin were purchased from Wisent Bioproducts (Saint-Jean-Baptiste, Canada).

### Synthesis of tetrahydro-2H-pyran-2-yl 2-bromoacetate (THP-Br)

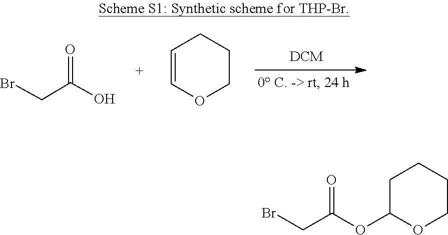 [SMILES: O=C(O)CBr ; C1=COCCC1 ; O=C(CBr)OC1CCCCO1]

To a dry round-bottom flask was added a stir bar, 2.0 g bromoacetic acid (14 mmol), and 7.2 mL DCM, dried over molecular sieves. The flask was purged with N₂ and cooled to 0° C. in an ice-water bath. 1.6 mL 3,4-dihydro-2H-pyran (17.2 mmol) was added dropwise via syringe. The reaction proceeded at room temperature (RT) for 24 h under N₂. The reaction mixture was transferred to a separatory funnel and washed 5 times with 5% NaHCO₃ followed by once with brine. The organic phase was dried over MgSO₄ and concentrated by rotary evaporation. The resulting clear, colourless oil (1.275 g, 41%) was used without further purification. MS (ESI⁺): m/z calculated for [M+H⁺] C₇H₁₂BrO₃⁺: 223.00, found: 223.13. ¹H NMR (400 MHz, CDCl₃): δ 5.31 (t, 1H), 3.93 (td, 1H), 3.87 (s, 2H), 3.73 (dtd, 1H), 1.9-1.4 (m, 6H).

### Synthesis of 1-ethoxyethyl 2-bromoacetate (EOE-Br)

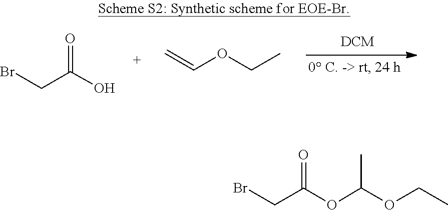

To a dry round-bottom flask was added a stir bar, 2.0 g bromoacetic acid (14 mmol), and 7.2 mL DCM, dried over molecular sieves. The flask was purged with N₂ and cooled to 0° C. in an ice-water bath. 1.6 mL ethyl vinyl ether (17.2 mmol) was added dropwise via syringe. The reaction proceeded at RT for 24 h under N₂. The reaction mixture was transferred to a separatory funnel and washed 4 times with 5% NaHCO₃ and once with brine. The organic phase was dried over MgSO₄ and concentrated by rotary evaporation. The resulting clear, colourless oil (1.494 g, 51%) was used without further purification. ¹H NMR (400 MHz, CDCl₃): δ 5.96 (q, 1H), 3.83 (d, 2H), 3.76 (dq, 1H), 3.56 (dq, 1H), 1.43 (d, 3H), 1.21 (t, 3H).

### Synthesis of methoxymethyl 2-bromoacetate (MOM-Br)

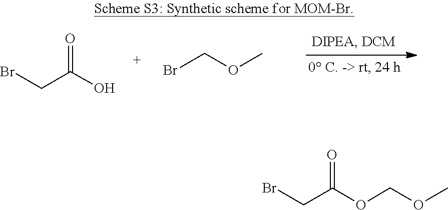

To a dry round-bottom flask was added a stir bar, 2.0 g bromoacetic acid (14 mmol), and 3.6 mL DCM, dried over molecular sieves. The flask was purged with N₂ and cooled to 0° C. in an ice-water bath. 2.76 mL diisopropylethylamine (15.4 mmol) was added dropwise via syringe. In a separate vial, 1.600 mL bromomethyl methyl ether (17.2 mmol) was dissolved in 3.6 mL DCM. This solution was added dropwise to the bromoacetic acid solution with stirring. The reaction was stirred at 0° C. for 2 h, then RT for 22 h under N₂. The reaction mixture was transferred to a separatory funnel and washed 5 times with pH 7.4 phosphate buffer (100 mM) followed by once with brine. The organic phase was concentrated by rotary evaporation. The resulting light brown oil (912 mg, 36%) was used without further purification. ¹H NMR (400 MHz, CDCl₃): δ 5.30 (s, 2H), 3.86 (s, 2H), 3.50 (s, 3H). ¹³C NMR (400 MHz, CDCl₃): δ 167.01, 92.02, 58.08, 25.77.

The reaction mixture turned cloudy as the reaction progressed. The polymer was then precipitated in ice-cold diethyl ether and the precipitate was pelleted by centrifugation at 4° C. (4500×g, 5 min). The pellet was then redissolved in minimal trifluoroethanol (TFE), and the precipitation and pelleting procedure was repeated twice more. The pellet was dried overnight in vacuo to produce an off-white to light brown solid. The product was characterized by ¹H NMR.

PLA-b-PCB-THP: ¹H NMR (500 MHz, DMSO-d₆): δ 7.61 (br, 1H), 6.04 (br, 1H), 5.17 (m, 1H PLA), 4.81 (br, 2H), 4.20 (br, 2H), 3.10 (br, 2H), 2.81 (br, 6H), 2.01-1.67 (br, 6H), 1.46 (br, 3H PLA), 1.05-0.71 (br, 5H). ¹H NMR (500 MHz, CD₃CN:D₂O=2:1): δ 7.70 (br, 1H), 6.06 (br, 1H), 5.07 (br, 1H PLA), 3.75 (br, 2H), 3.51 (br, 2H), 3.23 (br, 2H), 3.11 (br, 6H), 3.05 (br, 2H), 2.79 (br, 2H), 1.84 (br, 4H), 1.42 (br, 3H PLA), 1.31-1.21 (br, 6H), 1.05-0.71 (br, 5H).

PLA-b-PCB-EOE: ¹H NMR (500 MHz, DMSO-d₆): δ 7.57 (br, 1H), 6.05 (br, 1H), 5.18 (br, 1H PLA), 4.64 (br, 2H), 3.87 (br, 2H), 2.79 (br, 6H), 1.84 (br, 4H), 1.46 (br, 3H PLA), 1.31-1.21 (br, 6H), 1.05-0.71 (br, 5H). ¹H NMR (500 MHz, CD₃CN:D₂O=2:1): δ 7.70 (br, 1H), 6.06 (br, 1H), 5.07 (br, 1H PLA), 3.75 (br, 2H), 3.51 (br, 2H), 3.23 (br, 2H), 3.11 (br, 6H), 3.05 (br, 2H), 2.79 (br, 2H), 1.84 (br, 4H), 1.42 (br, 3H PLA), 1.31-1.21 (br, 6H), 1.05-0.71 (br, 5H).

PLA-b-PCB-MOM: ¹H NMR (500 MHz, DMF-d₇): 6.23 (br, 2H), 5.27 (br, 1H PLA), 4.78-4.56 (br, 2H), 3.28 (br, 6H), 3.11 (br, 6H), 1.97 (br, 2H), 1.56 (br, 3H PLA), 1.45-0.83 (br, 5H). ¹H NMR (500 MHz, CD₃CN:D₂O=1:1): δ 7.70 (br, 1H), 5.07 (br, 1H PLA), 3.75 (br, 2H), 3.51 (br, 2H), 3.23 (br, 2H), 3.11 (br, 6H), 3.05 (br, 2H), 2.79 (br, 2H), 1.84 (br, 4H), 1.42 (br, 3H PLA), 1.31-1.21 (br, 6H), 1.05-0.71 (br, 5H).

### Polymer Synthesis

PLA-b-PDMAPMA was synthesized as previously described.^{[33]} To synthesize PLA-b-PCB derivatives, PLA-b-PDMAPMA was dissolved in DMF at 75 mg/mL and cooled in an ice-water bath. Then, one of tetrahydro-2H-pyran-2-yl 2-bromoacetate (THP-Br), 1-ethoxyethyl 2-bromoacetate (EOE-Br) or methoxymethyl 2-bromoacetate (MOM-Br), equal to 3 molar equivalents of reactive amines on PLA-b-PDMAPMA, was added to the solution and stirred for 2 h in an ice-water bath.

### Synthesis of DSPE-CTA

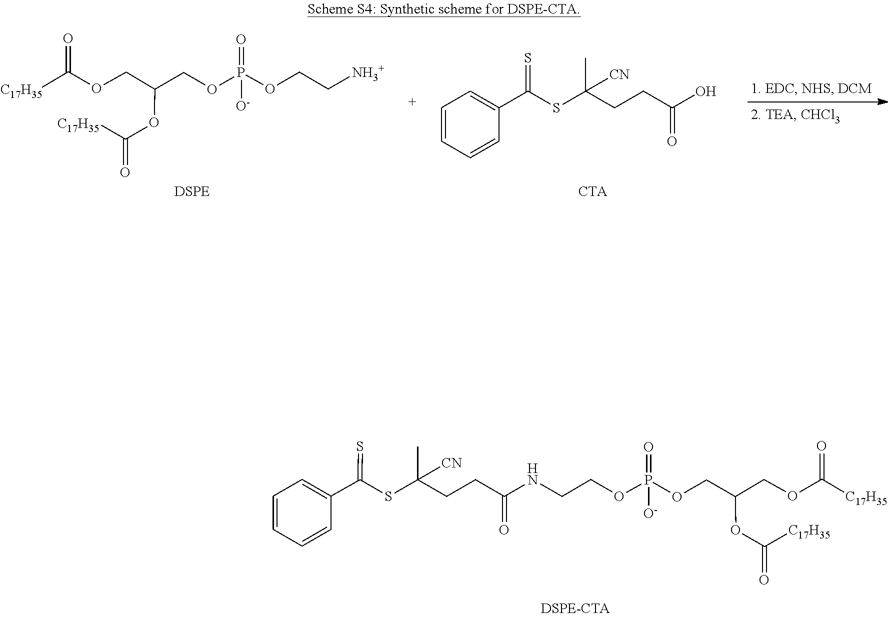

First, the carboxylic acid on the chain transfer agent (CTA) was activated with N-hydroxysuccinimide (NHS) to produce CTA-NHS. To a clean round-bottom flask was added 200 mg 4-cyano-4-((phenylcarbonothioyl)thio)pentanoic acid (0.70 mmol), 99 mg N-hydroxysuccinimide (NHS, 0.86 mmol), 165 mg 1-Ethyl-3-[3-dimethylaminopropyl]carbodiimide hydrochloride (EDC, 0.86 mmol), and 2.0 mL DCM, dried over molecular sieves. The flask was sealed and the reaction was stirred at RT overnight. A precipitate formed overnight, which dissolved with additional DCM. TLC of the reaction mixture (SiO₂; hexanes:ethyl acetate of 7:3, v/v) showed no more unreacted CTA. The reaction mixture was washed 3 times with ddH₂O and once with brine, then the organic layer was dried with MgSO₄ and filtered into a pre-weighed amber vial. After blowing off the solvent with N₂, 211 mg pink solid (CTA-NHS) was obtained and used without further purification (78%). MS (ESI⁺): m/z calculated for [M+H⁺] C₁₇H₁₇N₂O₄S₂⁺: 377.06, found: 377.06 [M+H⁺], 399.04 [M+Na⁺], 262.04 [C₁₃H₁₂NOS₂⁺], 121.01 [C₇H₅S⁺]. ¹H NMR (400 MHz, CDCl₃): δ 7.93 (dt, 2H), 7.58 (tt, 1H), 7.41 (tt, 2H), 2.99 (m, 2H), 2.85 (s, 4H), 2.75 (m, 1H), 2.57 (m, 1H), 1.96 (s, 3H).

To produce DSPE-CTA, 200 mg DSPE (0.26 mmol), 111.7 μL triethylamine (TEA, 0.78 mmol), and 16 mL CHCl₃ were added to a clean round bottom flask. All components dissolved with heating to 55° C. and stirring. The flask was cooled to RT and 100 mg CTA-NHS (0.26 mmol) was added. The flask was sealed and the reaction mixture was stirred for 2 h at 35° C. The reaction mixture was washed 3 times with 1 M HCl, once with ddH₂O and once with brine. The organic layer was dried with MgSO₄, then concentrated by blowing off the solvent with air. The concentrate was further purified by SiO₂ column chromatography. Unreacted CTA-NHS was eluted first using 99:1 v/v CHCl₃:EtOH, then the desired product was eluted in 80:20 v/v CHCl₃:EtOH. The fractions were combined and concentrated by rotary evaporation, producing 82 mg pink solid (30%). MS (ESI⁺): m/z calculated for [M+H⁺] C₅₄H₉₄N₂O₉PS₂⁺: 1009.6, found: 1009.6. ¹H NMR (400 MHz, CDCl₃): δ 7.89 (d, 2H), 7.53 (t, 1H), 7.36 (t, 2H), 7.04 (br, 1H), 5.23 (br, 1H), 4.37 (br, 1H), 4.15 (br, 1H), 3.95 (br, 4H), 3.48 (br, 2H), 2.61 (br, 2H), 2.40 (br, 2H), 2.27 (br, 4H), 1.91 (s, 3H), 1.55 (br, 4H), 1.25 (br, 48H), 0.88 (t, 6H).

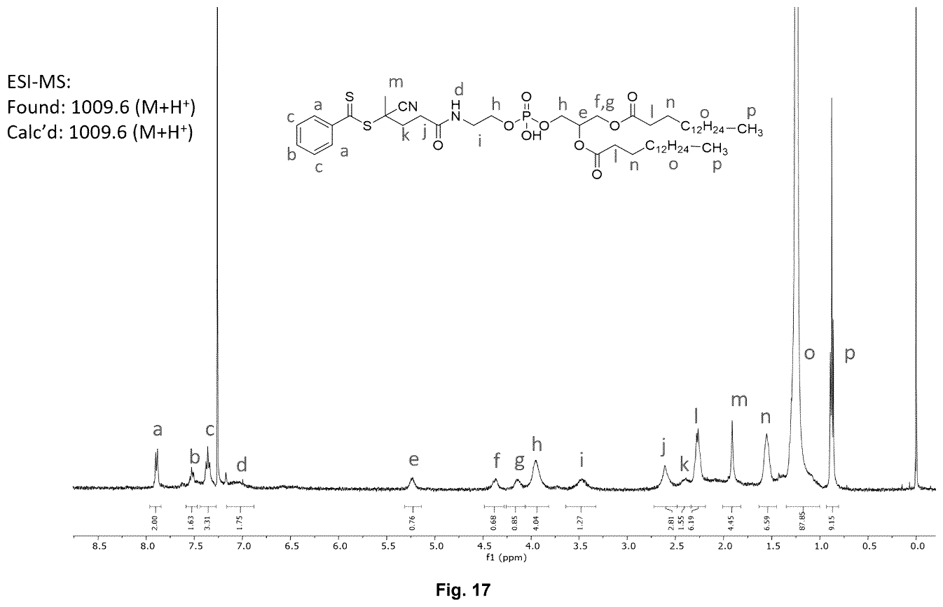

¹H NMR data are shown in FIG. 17.

### Synthesis of DSPE-PDMAPMA

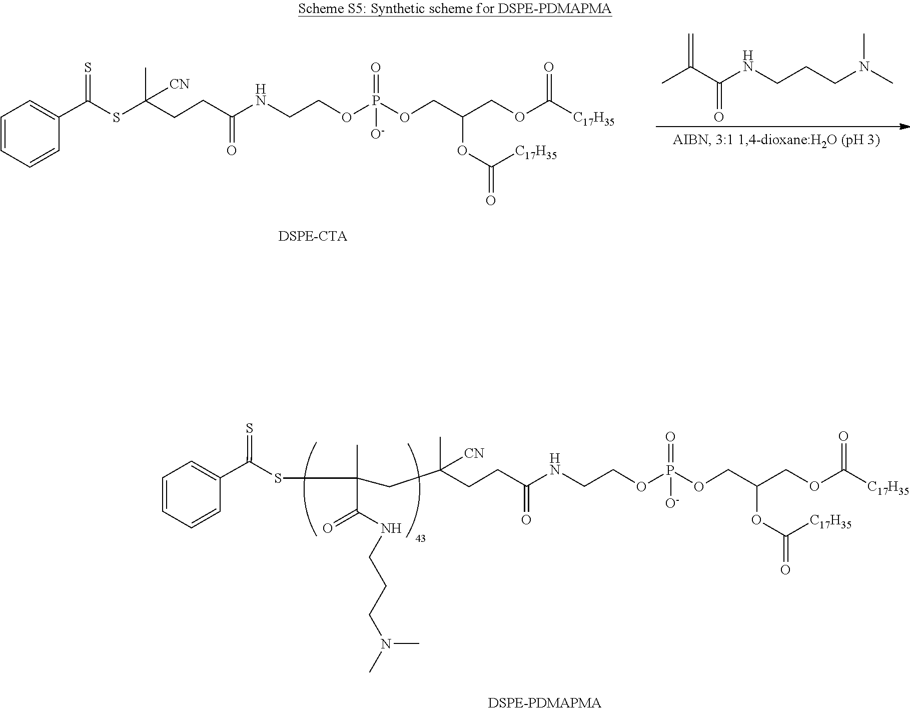

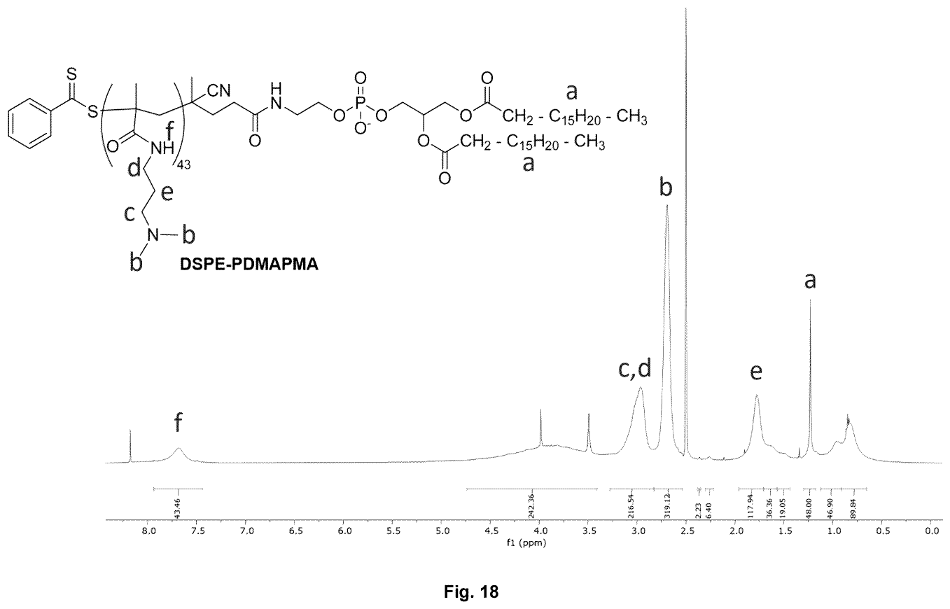

The following procedure is for a polymer with target Mn=6000 g/mol and CTA: initiator mole ratio of 3:1. To a round bottom flask was added 28 mg DSPE-CTA, 1.6 mg 2,2′-azobis(isobutyronitrile) (AIBN), and 1.5 mL 1,4-dioxane. Separately, N-(3-(dimethylamino)propyl)methacrylamide (DMAPMA) monomer was purified by flashing through a basic alumina plug. 157.9 μL purified monomer was dissolved in 0.500 mL H₂O, and 72.8 μL 12 M HCl was added to adjust pH. This solution was added to the 1,4-dioxane solution to produce a slightly cloudy 3:1, v/v dioxane: H₂O solution at pH 3. The solution was degassed by bubbling with N₂ for 30 min, then the flask was sealed and stirred overnight at 65° C. under N₂. The next morning, a gel-like substance was found at the bottom of the flask, which dissolved in H₂O. The entire reaction mixture was transferred to 1 kDa MWCO dialysis tubing, then dialyzed against ddH₂O for 24 h, changing the dialysis water 3 times. The dialyzed polymer was then frozen and lyophilized to produce 118 mg light pink-orange solid. The Mn was calculated by ¹H NMR by comparing the —N(CH₃)₂ peak at δ 2.69 ppm to the 48 H lipid peak at 1.24 ppm (FIG. 18). ¹H NMR (500 MHz, DMSO-d₆): δ 7.68 (br, 1H), 3.86 (br, 2H), 2.97 (br, 2H), 2.69 (br, 6H), 1.77 (br, 2H), 1.24 (s, DSPE 48H), 1.07-0.68 (br, 5H).

### Synthesis of DSPE-PCB Polymers

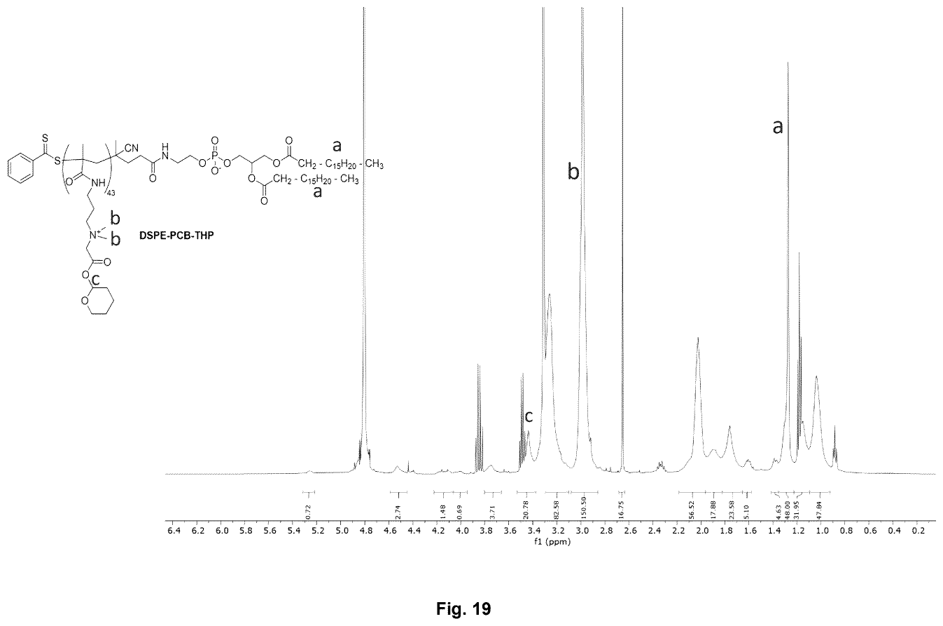

25 mg of DSPE-PDMAPMA polymer was added to a 1-dram vial and dissolved in 1.000 mL DMSO with heating and sonication. To produce DSPE-PCB-THP, DSPE-PCB-EOE and DSPE-PCB-MOM polymers respectively, 2 molar equivalents of THP-Br, EOE-Br, and MOM-Br to reactive amines were added to the vials. The vials were capped and agitated on a shaker at RT for 1.5 h. The polymers were then precipitated in ice-cold diethyl ether and pelleted by centrifugation (4500×g, 5 min, 4° C.). The pellets were redissolved in minimal 2,2,2-trifluoroethanol (TFE), then reprecipitated in diethyl ether and pelleted by centrifugation, as described. The process was repeated for dissolution, precipitation and pelleting, which were then dried in a vacuum oven at RT. DSPE-PCB-THP: 21 mg pink-orange solid obtained. DSPE-PCB-EOE: 21 mg pink-orange solid obtained. DSPE-PCB-MOM: 25 mg light brown solid obtained. DSPE-PCB-THP: ¹H NMR (500 MHz, CD₃OD): δ 7.68 (br, 1H), 3.26 (br, 2H), 2.98 (br, 6H), 2.03 (br, 2H), 1.95-1.56 (br, 6H), 1.76 (br, 2H), 1.27 (s, DSPE 48H), 1.19-0.94 (br, 5H) (FIG. 19).

DSPE-PCB-EOE: ¹H NMR (500 MHz, CD₃OD): δ 7.68 (br, 1H), 3.26 (br, 2H), 2.98 (br, 6H), 2.03 (br, 2H), 1.76 (br, 2H), 1.40 (br, 3H), 1.27 (s, DSPE 48H), 1.17 (br, 3H), 1.12-0.99 (br, 5H).

DSPE-PCB-MOM: ¹H NMR (500 MHz, CD₃OD): δ 7.68 (br, 1H), 3.43 (br, 3H), 3.26 (br, 2H), 2.98 (br, 6H), 2.03 (br, 2H), 1.76 (br, 2H), 1.27 (s, DSPE 48H), 1.06 (br, 5H).

### Polymer Nanoparticle Formulation

To produce polymer-only nanoparticles for gel retardation assay and zeta potential measurement, the PLA-b-PCB polymers were dissolved in TFE at 4 mg/ml. 125 μL polymer solution was then added to 375 μL PBS and mixed thoroughly by pipette. The solution was transferred to an Amicon Ultra 4 centrifugal filter unit (MWCO=10 kDa) (Millipore Sigma, Burlington, USA) and diluted further with 4 mL PBS. The NP suspension was concentrated by centrifugation (3500×g, 20 min, room temperature, RT) and resuspended in 500 uL PBS to produce a ˜1 mg/mL suspension.

### Gel Retardation Assay

Polymer NPs were concentrated using Amicon Ultra 0.5 centrifugal filter units (MWCO=10 kDa) and re-diluted to 2 mg/mL in PBS. Separately, siFLuc was diluted to 0.2 mg/mL in TBE buffer (90 mM Tris, 90 mM borate and 2 mM EDTA, pH 8.3). In a microcentrifuge tube, 10 μL NP suspension, 1.26 μL siRNA solution, and 3.74 μL TBE buffer were mixed to give an N:P ratio of 10. The mixtures were incubated 30 min at RT to allow for RNA complexation with NPs. 3 μL 6× loading dye was added to each mixture then loaded onto separate wells of 6% TBE SDS-PAGE gels (Thermo-Fisher, Waltham, USA). Electrophoresis was run in TBE buffer for 1 h at 100 V. Gels were stained in 2× SYBR Safe stain (Thermo-Fisher, Waltham, USA) in TBE buffer, then imaged under blue light using a E-Gel Imager system (Thermo-Fisher, Waltham, USA). Band intensities were calculated using ImageJ software and RNA was quantified against a calibration curve of free siFLuc.

### PLNP Formulation

Appropriate amounts of DSPC, cholesterol, and DMG-PEG₂ₖ were dissolved in ethanol such that they would compose 19, 25, and 6 wt % respectively of the PLNP. Amounts of PLA-b-PCB-X polymer and DLin-MC3-DMA varied between 0-50 wt % depending on the formulation. To aid solubility of the PLA-b-PCB-X polymer, a 10 mg/mL stock solution of polymer in TFE was produced first, then the appropriate amount was added to the lipid solution in ethanol. The total mass of lipids and polymer in the organic phase was 0.5 mg. Additional ethanol was added to give a total volume of 200 μL for the organic phase. Separately, the appropriate amount of RNA was dissolved in 25 mM acetate buffer (pH 4.0) to give the desired N:P ratio of 6 for siRNA or 10 for mRNA. The aqueous phase was brought to 600 μL in volume by adding acetate buffer.

For siRNA containing PLNPs, the organic and aqueous phases were mixed using a NanoAssemblr Benchtop microfluidic mixer (Precision Nanosystems, Vancouver, Canada). The phases were mixed at 3:1 aqueous: organic ratio with total flow rate 9 mL/min. For mRNA-containing PLNPs, the phases were mixed by pipetting up and down at least 15 times, then letting incubate at RT for 5 min. After mixing, the PLNPs were diluted to 4.5 mL in PBS, then transferred to Amicon Ultra 4 centrifugal filter units (MWCO=10 kDa). The PLNPs were concentrated by centrifugation (3500×g, 20 min, RT) then diluted back to 0.5 mL in PBS. The PLNPs were then sterilized by filtering through Millex GV 0.22 μm PDVF syringe filters, then stored at 4° C. until use.

### PLNP Characterization

PLNP size was determined by dynamic light scattering on either a Dynapro Plate Reader II (Wyatt Technology, Santa Barbara, CA) or Zetasizer Nano ZS (Malvern Panalytical, Malvern, United Kingdom). PLNP samples were diluted to 50 μg/mL in PBS and measured for at least 20 runs in duplicate measurements. Sizes are reported as intensity-weighted Z-averages.

Zeta potential was determined using a Zetasizer Nano ZS. PLNP samples ˜1 mg/mL in PBS were diluted 20× in ddH₂O, then transferred to a disposable zeta potential cell for measurement. Zeta potentials are reported as the mean of triplicate measurements.

RNA encapsulation was determined by RiboGreen assay, which measures free RNA in solution. Free RNA content was measured before and after disrupting PLNPs with 2% Triton X-100 and 2 mg/mL heparin in TE buffer, and % encapsulation was determined as:

$$\% encapsulation = ( 1 - \frac{{c}_{RNA , free}}{{c}_{RNA , disrupted}} ) * 100 \% \qquad ( 1 )$$

where c_{RNA,free} and c_{RNA, disrupted} represent the amounts of RNA detected before and after PLNP disruption by Triton and heparin.

For cryo-TEM, PLNPs were concentrated to at least 10 mg/mL in PBS using Amicon Ultra 0.5 centrifugal filter units (MWCO=10 kDa). 6 μL of this suspension was transferred to charged lacey formcar/carbon TEM grids (Ted Pella Inc., Redding, USA), and vitrified in liquid ethane using a Vitrobot system (Thermo Fisher, Waltham, USA). Grids were kept frozen in liquid nitrogen until imaging. Imaging was performed using a Talos L120C Transmission Electron Microscope (Thermo Fisher, Waltham, USA).

### Cell Culture

All cells were maintained in a humidified incubator at 37° C. and 5% CO₂. SK-OV-3, SK-OV-3-FLuc, SK-OV-3-mChG9, and T-47D cell lines were cultured in Corning T75 flasks in RPMI 1640 medium supplemented with 10% fetal bovine serum and 1% penicillin/streptomycin. HepG2 cells were maintained in Corning T75 flasks in DMEM supplemented with 10% fetal bovine serum and 1% penicillin/streptomycin. The medium was replaced twice per week and cells were passaged when they reached 75% confluency.

### FLuc Knockdown Assay

SK-OV-3-FLuc cells were seeded in white-walled, clear bottom 96-well plates at 5×10³ cells/well and allowed to adhere overnight. The culture medium was then removed and was replaced by 100 μL/well PLNP treatments in complete medium. Cells were incubated with treatments 24 h, then the treatments were removed, replaced with complete medium and incubated an additional 24 h. The medium was then replaced with 1× PrestoBlue (Thermo Fisher, Waltham, USA) in complete medium and the cells were returned to the incubator for 1 h. Fluorescence was read on a Tecan infinite TE200 plate reader (Männedorf, Switzerland) with excitation/emission 535/590 nm.

SteadyGlo reagent (Promega, Madison, USA) was reconstituted according to the manufacturer's instructions then diluted with an equal volume of HBSS. The PrestoBlue reagent was removed from cells and replaced with 50 L/well diluted SteadyGlo reagent. Cells were incubated 5 min at RT to allow for complete cell lysis, then luminescence was measured using the Tecan plate reader with 1 s integration time.

Metabolic activity was calculated by subtracting cell-free controls from all fluorescence measurements, then normalizing to untreated controls (Eqn. 2). FLuc knockdown was calculated by first dividing the luminescence measurements by the blank-subtracted fluorescence measurement from the same well. The normalized luminescence measurements were then normalized to untreated controls (Eqn. 3).

$$\% Metabolic activity = \frac{{Fluorescence}_{treatment} - {Fluorescence}_{cell - free}}{{Fluorescence}_{ctrl} - {Fluorescence}_{cell - free}} \qquad ( 2 )$$

$$\% Luminescence = \frac{\frac{{Luminescence}_{treatment}}{{Fluorescence}_{treatment} - {Fluorescence}_{cell - free}}}{\frac{{Luminescence}_{ctrl}}{{Fluorescence}_{ctrl} - {Fluorescence}_{cell - free}}} \qquad ( 3 )$$

FLuc mRNA Transfection Assay

SK-OV-3 cells were seeded in white-walled, clear-bottom 96-well plates at 1×10⁴ cells/well and allowed to adhere overnight. The medium was removed and replaced with 100 μL/well PLNP treatments in complete medium. The cells were incubated 24 h with treatments, then the treatments were replaced with complete medium and incubated another 24 h.

Metabolic activity was measured with PrestoBlue as described in the previous section. The PrestoBlue was then removed and the cells were washed once with PBS. The cells were then lysed with 55 μL Glo Lysis Buffer (Promega, Madison, USA). 30 μL cell lysate was then transferred to a clear 96-well plate and the total protein content was measured using Pierce BCA Protein Assay Kit (Thermo Fisher, Waltham, USA) according to the manufacturer's instructions. To the remaining 25 μL cell lysate was added 25 μL reconstituted SteadyGlo reagent. After 5 min incubation at RT, luminescence was measured on a plate reader with 1 s integration time. Luminescence (in RLU) was normalized to total protein content.

### CCNE1 Knockdown Assay

Palbociclib-resistant T-47D cells were seeded in 24-well plates at 1×10⁵ cells/well and allowed to adhere overnight. The medium was removed and replaced with 500 μL/well PLNP treatments in complete medium. The cells were incubated 24 h with treatments, then replaced with 500 μL/well complete medium, and incubated another 24 h. The cells were then detached using trypsin-EDTA, pelleted and resuspended in PBS. The cells were then pelleted once more, flash-frozen in liquid nitrogen and stored at −80° C. until RNA extraction.

Total RNA was extracted from cell pellets using NucleoSpin RNA II kit (Macherey-Nagel, Düren, Germany) according to the manufacturer's instructions. RNA was quantified using the RNA-40 module on a Nanodrop UV-Vis spectrometer (Thermo Fisher, Waltham, USA). cDNA was synthesized immediately after RNA extraction using Superscript VILO kit (Thermo Fisher, Waltham, USA) according to the manufacturer's instructions. cDNA was stored at −80° C. until qPCR.

DNA primers for CCNE1 and ACTB (50 UM) were diluted in Power UP SYBR Green Master Mix (Thermo Fisher, Waltham, USA) at a 1:10 ratio. 5.5 μL of this master mix was then mixed with 4.5 μL cDNA solution in each well of a 384-well qPCR plate. qPCR was performed using a QuantStudio 6 Real-Time PCR System (Thermo Fisher, Waltham, USA), with 40 cycles of 15 s denaturation at 95° C. and 1 min annealing/extension at 60° C. CCNE1 knockdown was quantified using the ΔΔC_{T} method with ACTB as the reference gene.

Cy3 siRNA Localization Studies

Cy3-siRNA was encapsulated in PLNPs using the protocol described above. SK-OV-3 cells were seeded in black 96-well plates at 2.5×10⁴ cells/well and allowed to adhere overnight. The medium was aspirated, then the cells were dosed with 50 μL/well of 25 nM Cy3-siRNA in complete medium. The cells were incubated with treatments for 3 h in a humidified 37° C. incubator. The cells were washed once with complete medium, then stained with 5 μg/mL Hoechst 33342 in complete medium 10 min at 37° C. The stain was replaced with complete medium, then immediately imaged using a Zeiss Apotome Live Cell AxioObserver inverted fluorescent microscope (Oberkochen, Germany). Cells were imaged live and maintained in a humidified 37° C. incubator with 5% CO₂ during imaging. A 20× Plan Neofluor objective (NA 0.4) (Carl Zeiss Canada) was used, and 4 images were taken in a 2×2 pattern and stitched together to produce a wide-angle composite image. Image acquisition was automated by setting the focal plane to the nuclei channel (Hoechst). The channels used were Hoechst (Ex/Em=359-371/>397 nm) and Cy3 (540-580 593-668). Typically, 100% laser power was used with exposure times ranging from 500-1500 ms. Exposure times and illumination were kept constant across all wells of a plate.

Cell counting and Cy3 intensity analysis were performed using ImageJ. In the Hoechst channel, a threshold was automatically applied to eliminate background signal, then the image was binarized and a watershed algorithm applied to separate touching cells. Particles >20 μm² were counted as cell nuclei. In the Cy3 channel, a threshold was automatically applied to eliminate background signal, then total Cy3 signal across the entire image was quantified using the RawIntDen function.

### Uptake and Endosomal Escape Determination by Confocal Microscopy

SK-OV-3 cells were transduced with mCherry-Galectin9 (mChG9) plasmids according to a previous protocol to produce the SK-OV-3-mChG9 cell line. Separately, PLNPs were stained by modifying the earlier procedure to include 0.1 wt % DiD (Thermo Fisher, Waltham, USA) in the organic phase during formulation. SK-OV-3-mChG9 cells were seeded in black-walled clear-bottom 96-well plates at 2.5×10⁴ cells/well and allowed to adhere overnight. The medium was removed and replaced with 50 μL of 10 nM siRNA in DiD-stained PLNPs. The cells were incubated with treatments for 3 h, then washed once with PBS and fixed with 4% paraformaldehyde in PBS for 15 min at RT. The fixative was removed, then the cells were stained with 5 μg/mL Hoechst 33342 in PBS for 15 min at RT. Cells were washed twice with PBS, and then replaced with 100 μL PBS and stored at 4° C. in the dark until imaging.

Cells were imaged on a Zeiss Apotome Live Cell AxioObserver inverted fluorescent microscope (Oberkochen, Germany) with a long working distance 40× Plan Neofluor objective (NA 0.6) (Carl Zeiss Canada), an X-Cite 120 LED fluorescent lamp (Lumen Dynamics), and an Axiocam 506 mono camera (Carl Zeiss Canada). Zen Blue 2.3 was used to acquire images. Image acquisition was automated by setting the focal plane to the nuclei channel (Hoechst). 9 images were taken in a 3×3 pattern and stitched together to produce a wide-angle composite image. The channels used were Hoechst (Ex/Em=359-371/>397 nm), mCherry (540-552/575-640 nm) and DiD (625-655/665-715 nm). Typically, 100% laser power was used with exposure times ranging from 500-3000 ms. Exposure times and illumination were kept constant across all wells of a plate.

NP and Gal9 foci were quantified using a MATLAB script modified from Kilchrist et al.^{[68]} which is available at: https://github.com/kaislaughter/mChG8_image_processing. The script first applied a threshold to eliminate background signal, then performed a top hat transform to identify cell nuclei, NP puncta, and Gal9 puncta. The images were then binarized and a watershed algorithm applied to separate touching features. Cell features were then automatically counted and tabulated. At least 100 cells were counted per image, and the average counts across 3-6 wells were considered as one biological replicate.

Uptake Studies with Endocytosis Inhibitors

SK-OV-3-mChG9 or T-47D cells were seeded in black 96-well plates at 2.5×10⁴ cells/well and allowed to adhere overnight. Genistein, nystatin, chlorpromazine, amiloride, and hydroxydynasore stock solutions were prepared in sterile DMSO. Immediately before dosing with cells, inhibitor solutions were prepared in complete medium using the stock solutions. The concentrations used to dose cells were 200 μM genistein, 25 μg/mL nystatin, 10 μg/mL chlorpromazine, 2.5 mM amiloride and 100 μM hydroxydynasore, in keeping with previously established protocols.^{[61,62]} The cells were incubated with 50 μL/well inhibitor solution for 30 min, then 50 μL 20 nM siRNA in PLNPs were added without removing the inhibitor solutions for a final concentration of 10 nM siRNA. The cells were incubated with the PLNPs and inhibitors for 3 h. Fixation, staining, image acquisition and image processing proceeded as described in the previous section.

### Statistical Analysis

All data are displayed as mean±standard deviation unless otherwise noted. Statistical analysis was performed using GraphPad Prism 8 software (San Diego, CA). Differences between two groups were tested for statistical significance using Student's t-test, while differences between more than two groups were tested using either one-way or two-way ANOVA with Tukey's post-hoc test unless otherwise noted. Where appropriate, a maximum of 1 outlier per group was removed using Grubbs' test (α=0.05).

### Results and Discussion

### Synthesis, Formulation and Characterization of Acid-Responsive Polymers

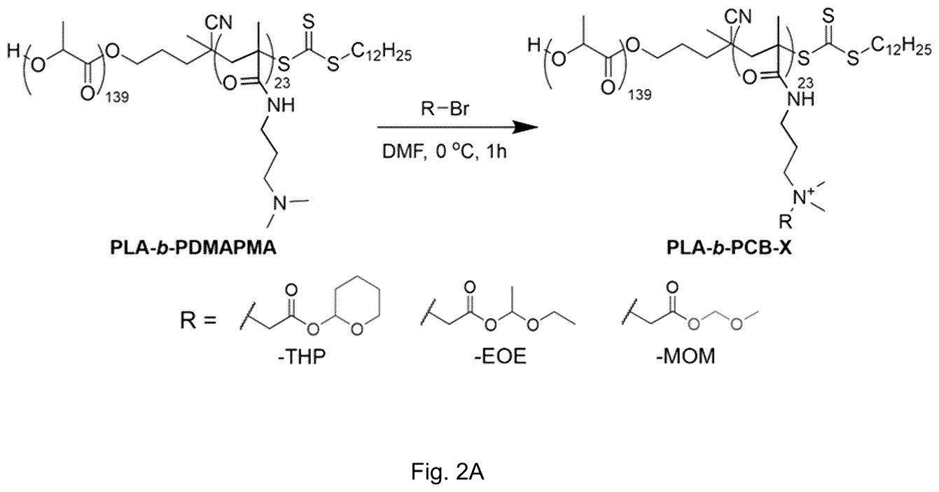

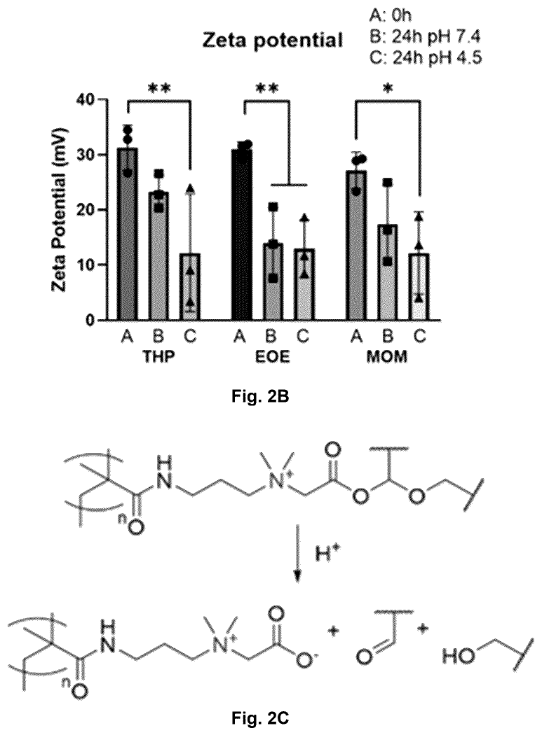

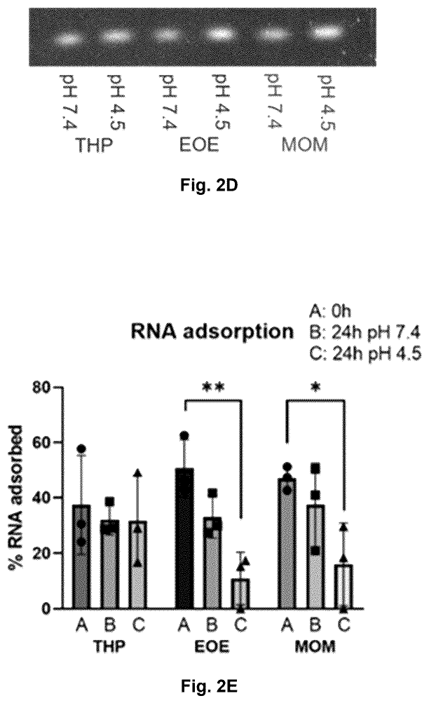

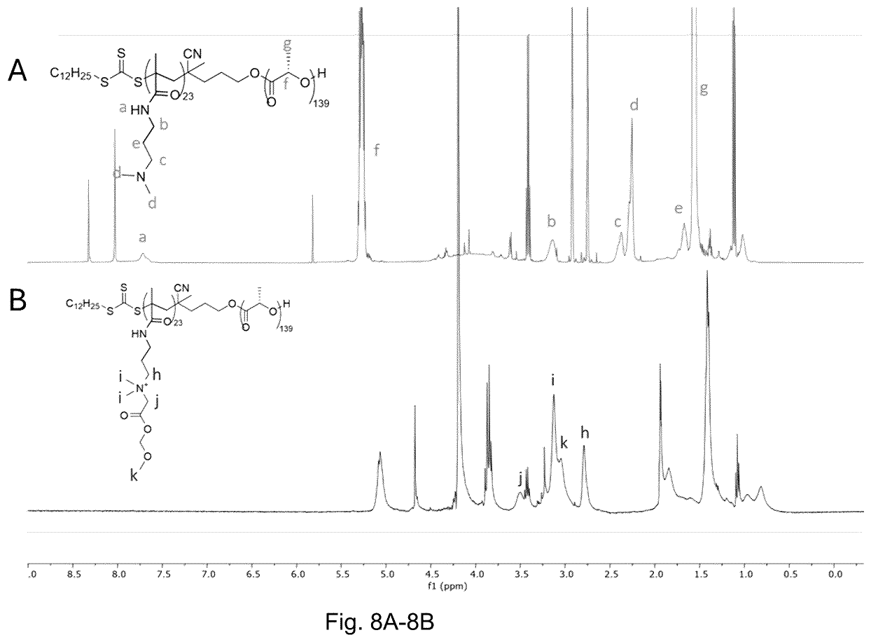

To synthesize the acid labile PLA-b-PCB-X, the inventors first synthesized the precursor polymer poly(lactic acid)-block-poly(N-[3-(dimethylamino)propyl]methacrylamide) (PLA-b-PDMAPMA) according to previous methods.^{[33]} The inventors then synthesized the hemiacetal ester small molecules, tetrahydro-2H-pyran-2-yl 2-bromoacetate (THP-Br), 1-ethoxyethyl 2-bromoacetate (EOE-Br) and methoxymethyl 2-bromoacetate (MOM-Br), and then used them to alkylate PLA-b-PDMAPMA (FIG. 2A).^{[29]} Alkylation of PLA-b-PDMAPMA by THP-Br, EOE-Br and MOM-Br resulted in amphiphilic, cationic block co-polymers PLA-b-PCB-THP, PLA-b-PCB-EOE, and PLA-b-PCB-MOM (FIG. 8A and FIG. 8B respectively): these hemiacetal ester groups impacted hydrolysis due to differences in both hydrophobicity and steric hindrance.

The inventors synthesized NPs by self-assembly of the polymer and characterized their zeta potential immediately on formulation and then 24 h later after incubation at 37° C. in either pH 7.4 or pH 4.5 citrate buffer. These pH values were chosen to represent the pH of serum and the lysosome, respectively,^{[41]} thereby providing some insight into the RNA-releasing capabilities of our polymers in acidic conditions. Immediately after formulation (FIG. 2B, 0 h), all 3 nanoparticles had strongly positive zeta potentials of approximately +30 mV, which supports the proposed chemical structures. The zeta potential of the NPs decreased to between +20 mV and +10 mV after 24 h incubation in pH 7.4 buffer, and more consistently to +10 mV, which is often considered close to neutral, in pH 4.5 buffer (FIG. 2B). Without being bound by any theory, the inventors attribute this effect to the increased hydrolysis of the pendant hemiacetal esters in acid, resulting in the carboxylate group which neutralizes the overall charge of the quaternary ammonium group in each monomer (FIG. 2C). This demonstrates that hydrolysis, and consequently charge neutralization, is more complete at lysosomal pH.

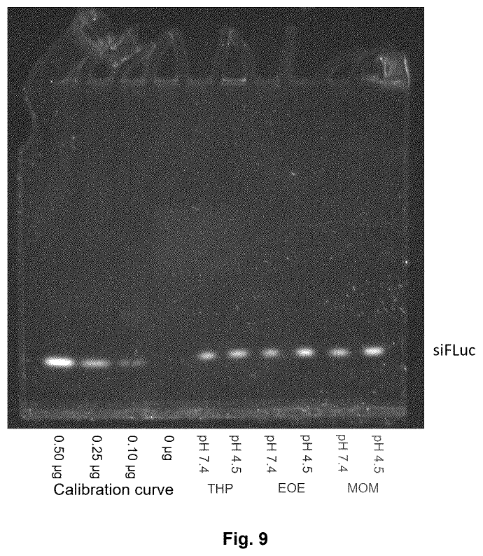

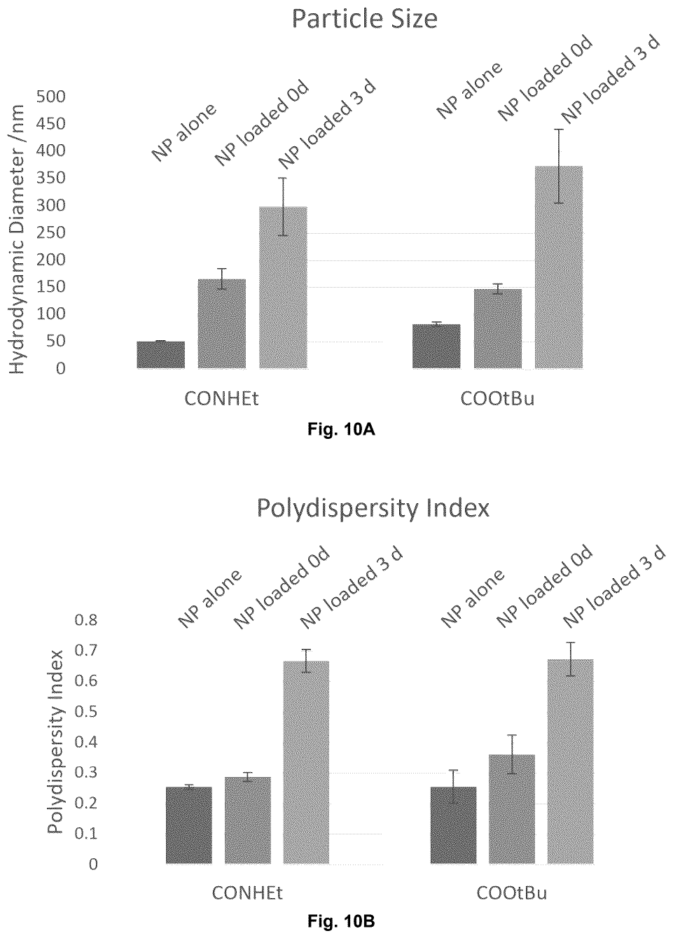

To determine if the reduced zeta potential correlates with RNA release, the inventors examined the ability of the hydrolyzed NPs to complex RNA using a gel retardation assay. The NPs, after 24 h incubation in either pH 7.4 or pH 4.5 citrate buffers, were incubated with siRNA at an N:P molar ratio of 10 for 30 min at room temperature (RT), and then any free RNA remaining was measured by gel electrophoresis. The NPs incubated at pH 4.5 had less RNA adsorbed to them than those at pH 7.4, as demonstrated by brighter and wider RNA bands, which reflect the charge neutralization of our NPs (FIG. 2D, full gel in FIG. 9). Specifically, PLA-b-PCB-EOE and PLA-b-PCB-MOM NPs adsorbed 60-70% less RNA at pH 4.5 than at pH 7.4 whereas PLA-b-PCB-THP NPs absorbed roughly the same amount of RNA at both pH values (FIG. 2E). The inventors hypothesize, without being bound by any theory, that the larger, more hydrophobic pendant groups of THP allow more RNA to remain complexed with the NPs by and electrostatic interactions, thus reducing release at acidic pH. Conversely, for smaller EOE and MOM pendant groups, the drop in zeta potential led to lower RNA complexation, which suggests that they would be good candidates for acid-responsive RNA release.

### Formulation and Characterization of PLNPs

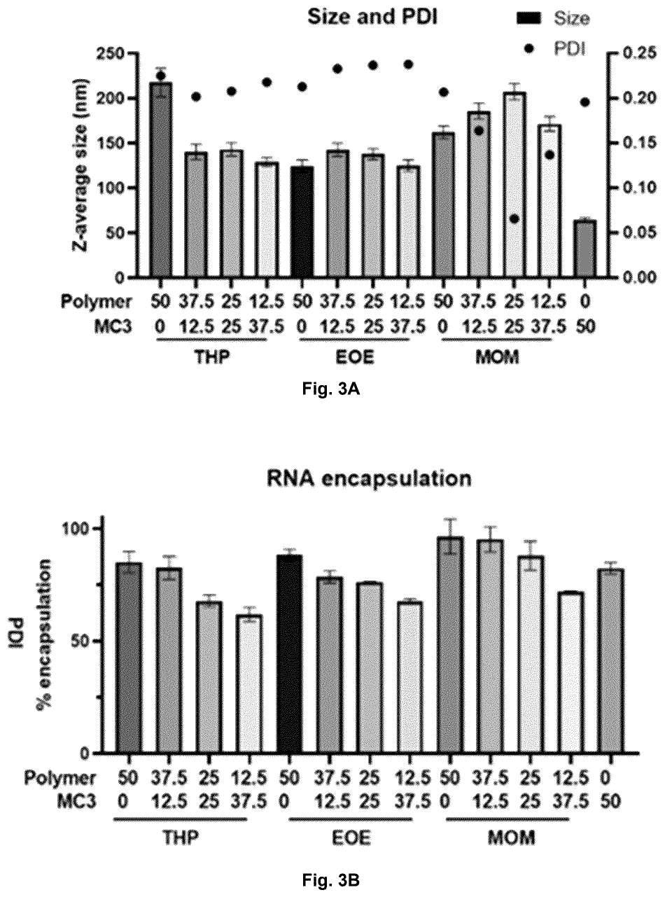

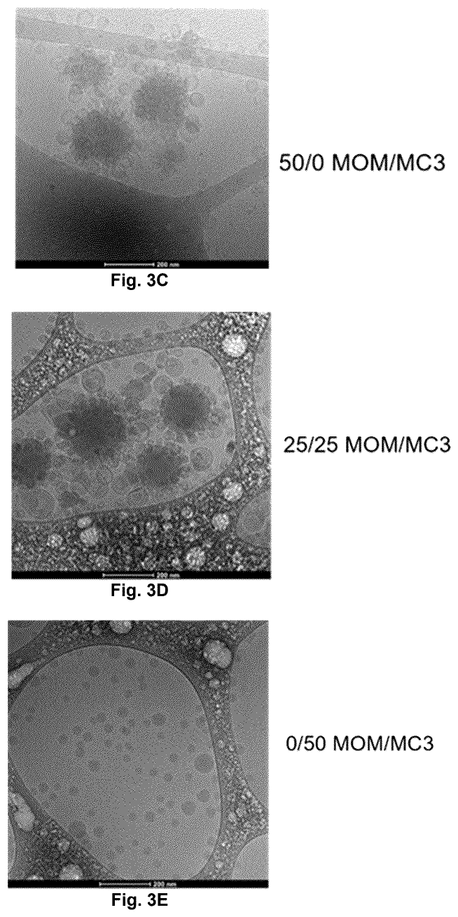

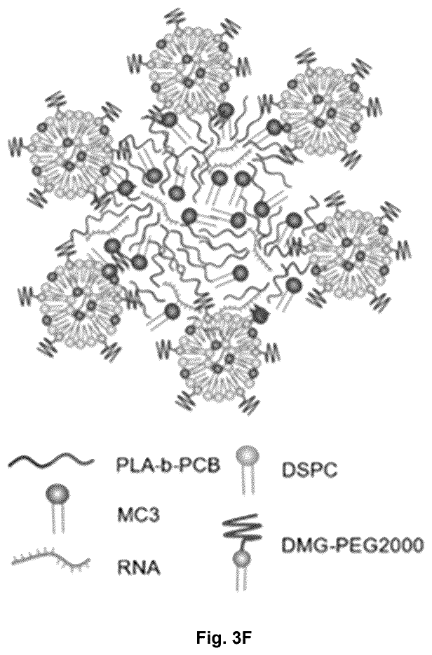

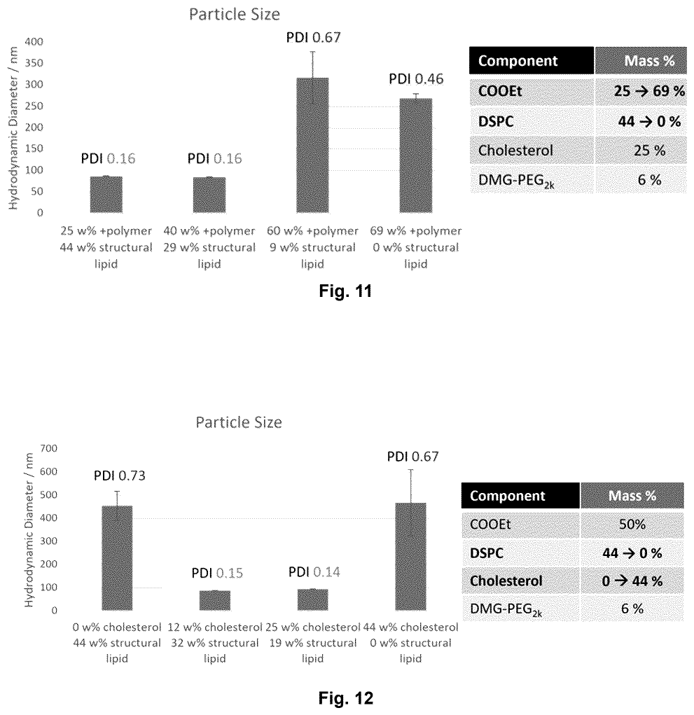

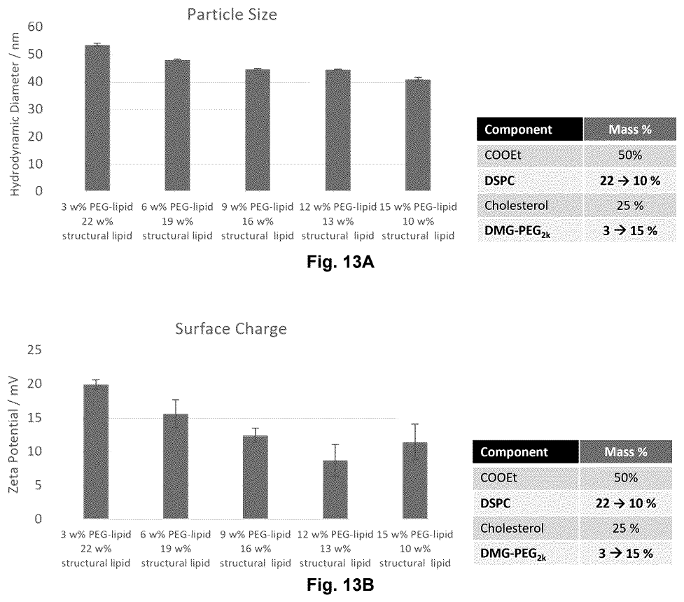

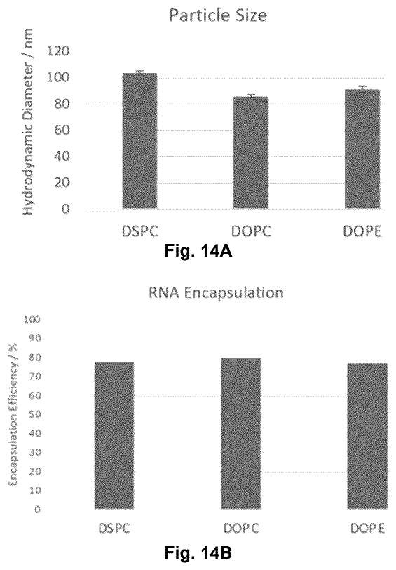

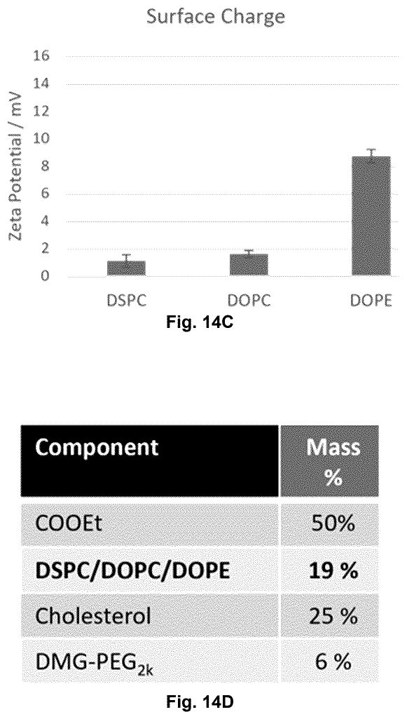

To improve RNA complexation efficiency and prevent particle aggregation, the inventors formulated our PLA-b-PCB-X polymers with lipids to produce PLNPs. Without lipids, the PLA-b-PCB-X NPs aggregated (FIGS. 10A-10B) and had poor RNA complexation efficiency, with only 30-60% RNA complexed at an N:P ratio of 10 (data not shown). These PLNP formulations were based on the clinically approved Onpattro formulation, which consists of MC3, DSPC, cholesterol and DMG-PEG_{2K}.^{[38]} Initially, the inventors replaced all of the MC3 with polymer and modified the proportions of remaining lipids to produce stable PLNPs. The inventors found that all lipid components were necessary, but the relative amounts could be altered without significant change in size of the PLNPs in most formulations (FIG. 11-14D). The inventors therefore maintained the proportion of DSPC, cholesterol and DMG-PEG_{2K} to 19, 25, and 6 wt %, respectively, and changed the remaining 50% of MC3 to include varying amounts of PLA-b-PCB-X polymer. The inventors rationalized that PLA-b-PCB-X was unlikely to induce endosomal escape on its own and thus a mixture of both MC3 and polymer would maximize both endosomal escape and RNA release into the cytosol. All PLNPs had z-average diameters of 100-200 nm with PDIs lower than 0.3, indicating relatively monodisperse populations (FIG. 3A). The sizes were larger than those of conventional MC3 LNPs (65 nm), indicating that the inclusion of PLA-b-PCB-X polymer, regardless of amount, increased particle size. As nanoparticles with diameters <200 nm are taken up by cells, the inventors proceeded to test our PLNPs in vitro. RNA encapsulation efficiency was between 62 and 96%, as measured by the RiboGreen assay, with a trend of greater RNA encapsulation with increasing PLA-b-PCB-X content (FIG. 3B).

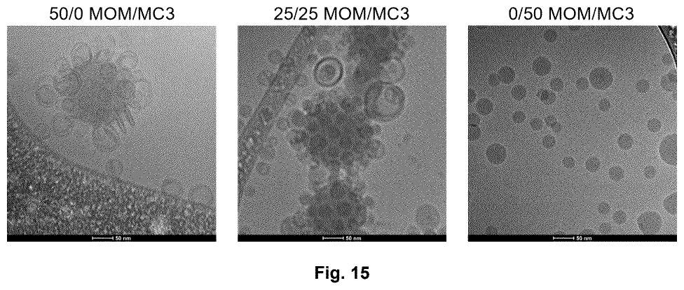

Since both the 50% MC3 LNP with no polymer and the 50% polymer PLNPs with no MC3 had good encapsulation efficiencies (>82%), the inventors wondered why mixing both polymer and MC3 in the same nanoparticle resulted in lower RNA encapsulation. The inventors hypothesized that polymer and MC3 were poorly mixed within these PLNPs, and thus examined the morphology of our PLNPs by cryo-TEM. The 50/0 MOM/MC3 PLNPs (i.e., no MC3) consisted of an amorphous core surrounded by hollow vesicle-like structures (FIG. 3C, additional images in FIG. 15). As PLA-b-PCB will self-assemble into amorphous core-shell structures,^{[33,42]} the amorphous core likely consists primarily of the PLA-b-PCB-MOM polymer. Additionally, as PLA-b-PCB-MOM is the only cationic species present in these PLNPs, the complexed RNA is also likely present in the amorphous area. Since DSPC, DMG-PEG_{2K} and cholesterol form highly organized bilayer structures, as previously determined by small-angle X-ray scattering and cryo-TEM studies of liposomal formulations,^{[43,44]} the inventors suggest that the hollow vesicle-like structures surrounding the amorphous core consist mainly of DSPC, DMG-PEG_{2K} and cholesterol. Some of these vesicles that do not appear to be associated with the polymer may indicate an excess of lipids in the formulations. Similar vesicle-like structures appeared when MC3 was introduced in 25/25 MOM/MC3 PLNPs (FIG. 3D), but some vesicles appeared to be filled in. The inventors attribute the filled-in vesicles to MC3 because unlike the other lipids, MC3 adopts an amorphous morphology at pH 7.4.^{[45]} This is consistent with our observations for 0/50 MOM/MC3 LNPs (i.e., no PLA-b-PCB-MOM) which appear as homogenous amorphous globules (FIG. 3E). The inventors also observed darkening of the amorphous core in the 25/25 MOM/MC3 formulation compared to that of 50/0 MOM/MC3, which suggests that some MC3 is also found in the amorphous core. This supports our hypothesis that RNA is found in the amorphous core, as both MC3 and PLA-b-PCB-MOM polymer are cationic and can complex RNA. The inventors also suggest that some RNA may be found in the filled-in vesicles of the 25/25 MOM/MC3 formulation, as the MC3 therein would likely complex RNA. Importantly, the conventional morphology of MC3 LNPs (i.e., 0/50 MOM/MC3, FIG. 3E are absent in the PLNP formulations, indicating that MC3 was successfully mixed into these formulations and did not form a separate population of LNPs. These observations led to the proposed structure for our PLNPs (FIG. 3F).

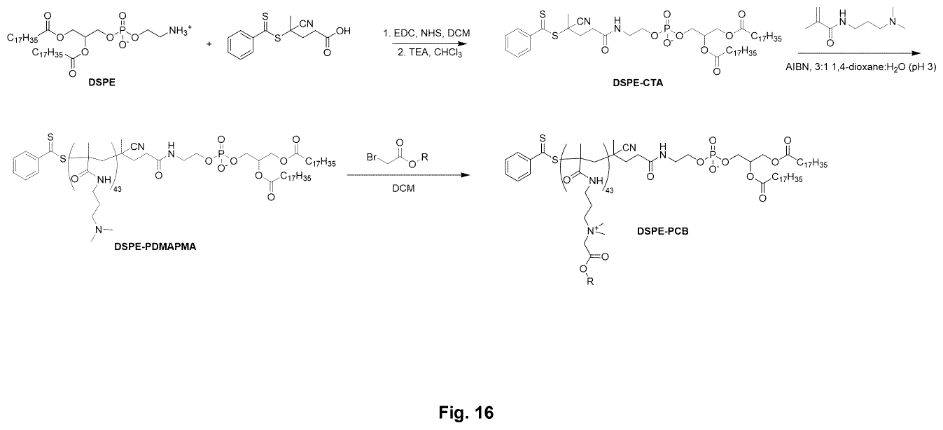

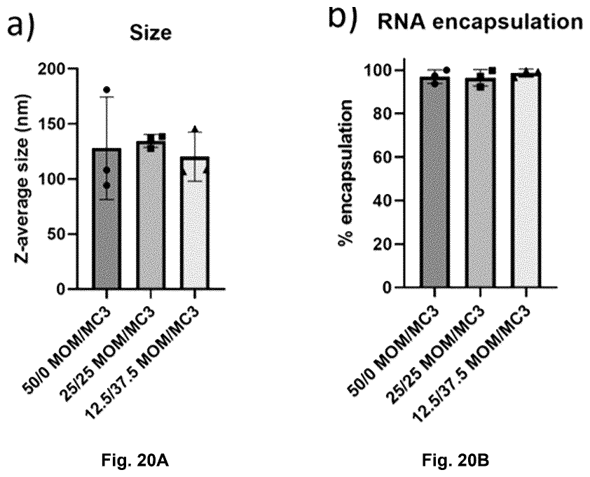

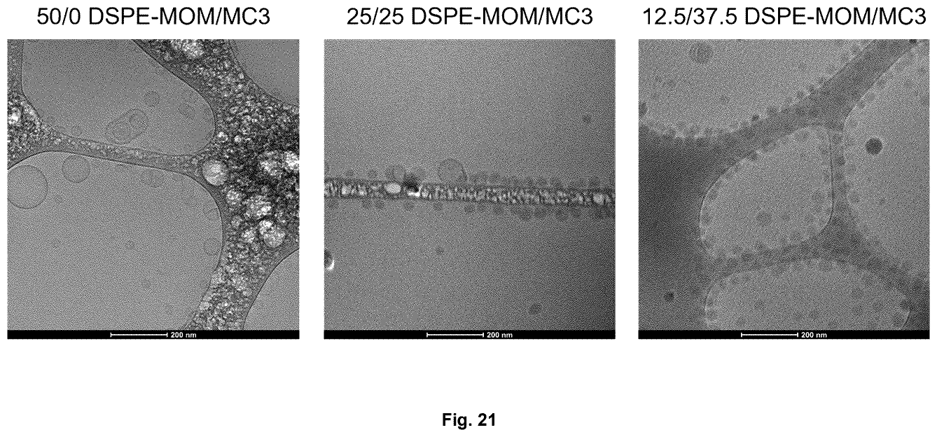

To determine whether the PLA or the PCB block were responsible for the dense amorphous core, the inventors also synthesized DSPE-PCB-X polymers with a lipid as the hydrophobic anchor instead of PLA (FIG. 16-19). The DSPE-PCB-X PLNPs had sizes between 100-150 nm (FIG. 20A), which were larger than conventional LNPs (65 nm). This suggests that the large size of the PCB block compared to the rest of the lipids contributes to the large size of PLNPs. In contrast to PLA-b-PCB-X PLNPs, DSPE-PCB-X PLNPs had encapsulation efficiencies of almost 100%, suggesting that poor mixing between PLA-b-PCB-X polymers and lipids was responsible for lower encapsulation efficiency (FIG. 20B). Cryo-TEM images of 50/0 DSPE-PCB-MOM/MC3 PLNPs (i.e. no MC3) consisted entirely of hollow vesicles without an amorphous solid core (FIG. 21) suggesting that the PLA block, and not the PCB, is responsible for the solid core in PLA-b-PCB-X PLNPs. The images also confirm that DSPC, cholesterol and DMG-PEG_{2K} make bilayer structures, consistent with previous reports. [46,47] The presence of MC3 in 25/25 DSPE-PCB-MOM/MC3 and 12.5/37.5 DSPE-PCB-MOM/MC3 resulted in filled-in, amorphous NPs with some degree of blebbing, likely resulting from excess DSPC and cholesterol, consistent with another system.^{[45]}

PLNPs Enhance siRNA-Mediated Knockdown

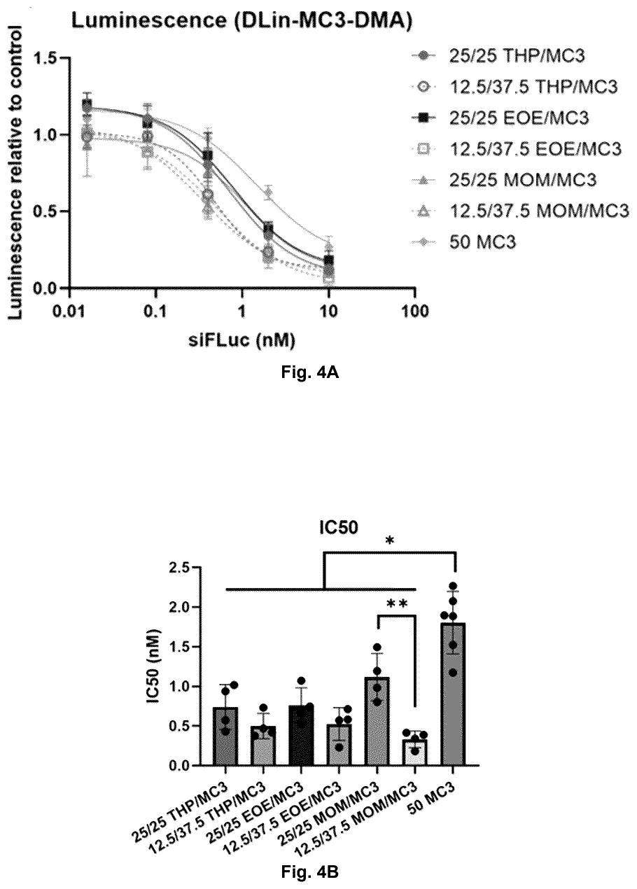

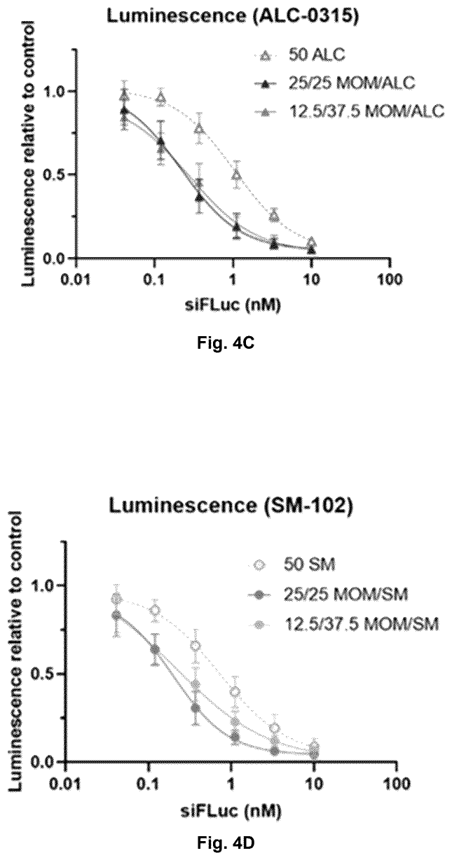

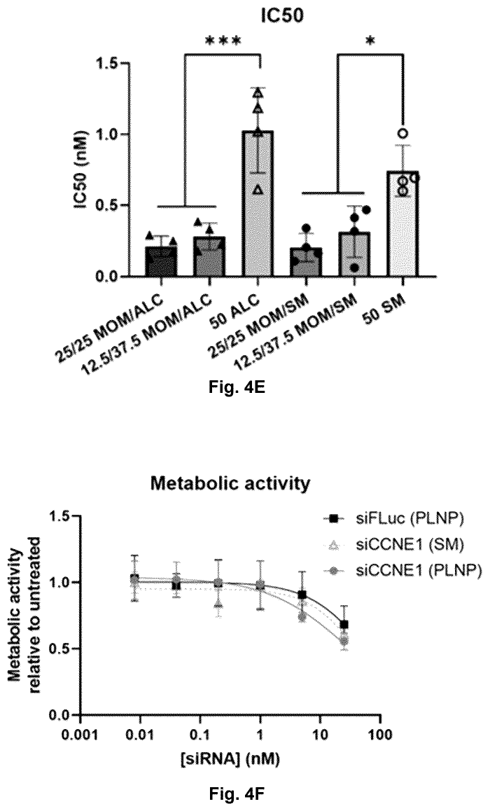

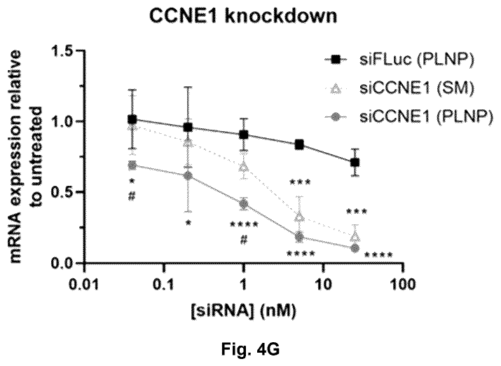

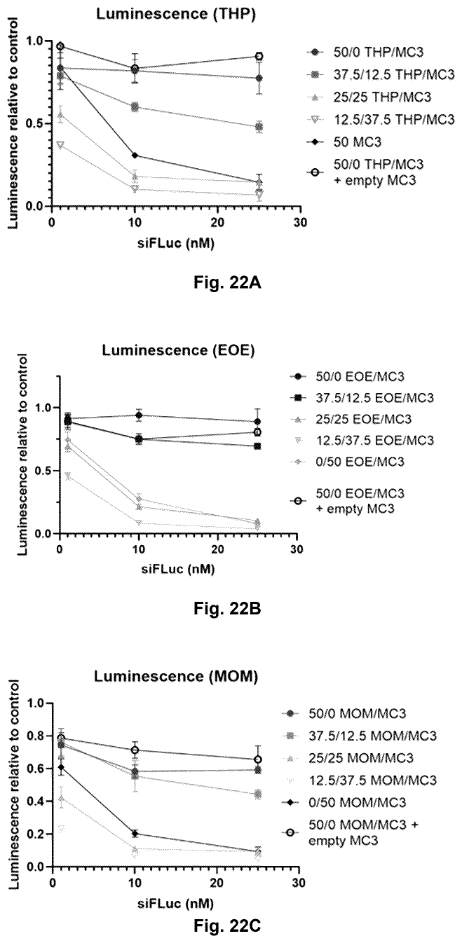

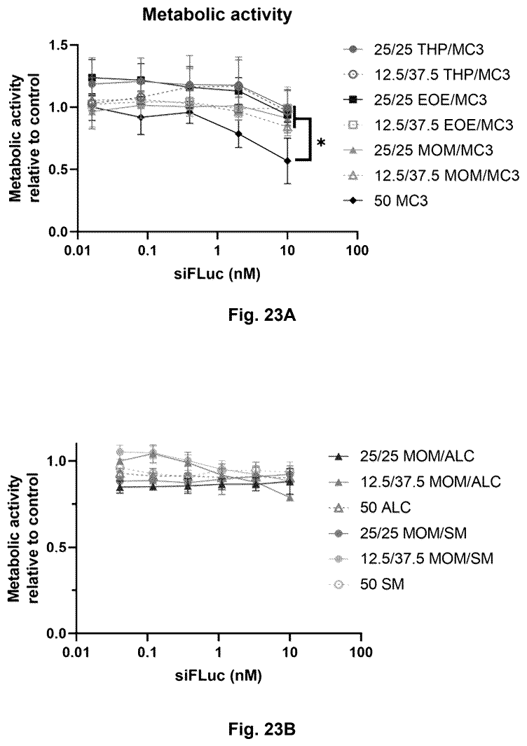

To examine the efficacy of our PLNPs in RNA delivery, the PLA-b-PCB-X PLNPs were used to encapsulate siRNA against firefly luciferase (siFLuc) and knockdown FLuc expression in an SK-OV-3-FLuc reporter cell line. To find the ratio of RNA-releasing polymer to ionizable lipid that would result in the greatest knockdown, the proportion of PLA-b-PCB-X/MC3 was varied from 50/0 to 0/50. Neither the 50/0 nor 37.5/12.5 PLA-b-PCB-X/MC3 PLNP formulations induced significant FLuc knockdown (FIG. 22A-22C), suggesting that a minimum amount of MC3 is required for significant knockdown. Moreover, simply co-delivering 50/0 PLA-b-PCB-X/MC3 PLNPs containing siFLuc along with empty MC3 LNPs (i.e., without siRNA) was also ineffective at knocking down RNA, indicating that the endosome disruption of MC3 must be incorporated in the PLNPs for effective RNA delivery (FIG. 22A-22C). Interestingly, the 25/25 and 12.5/37.5 PLA-b-PCB-X/MC3 PLNPs induced knockdown comparable to, or better than, conventional MC3 LNPs (FIG. 4A). Moreover, at the highest concentrations tested, all PLNPs were less toxic (as measured by PrestoBlue metabolic assay, FIG. 23A) than the 50 MC3 formulation, likely due to the lower amount of ionizable lipid. There were no significant differences between PLA-b-PCB-THP, -EOE, and -MOM with regards to toxicity or knockdown; however, the 12.5/37.5 PLA-b-PCB-MOM/MC3 formulation had an IC₅₀ 3-fold lower than that of the 25/25 PLA-b-PCB-MOM/MC3 (p<0.01, FIG. 4B). Almost all formulations had significantly lower IC₅₀ compared to that of 50 MC3, with the 12.5/37.5 PLA-b-PCB-MOM/MC3 formulation having the greatest knockdown with an IC₅₀ 5.4-fold lower.

Since the commercialization of Onpattro, additional ionizable lipids have been used clinically. Of note, Pfizer and Moderna developed [(4-Hydroxybutyl)azanediyl]di(hexane-6,1-diyl)bis(2-hexyldecanoate) (ALC-0315) and 9-Heptadecanyl8-{(2-hydroxyethyl) [6-oxo-6-(undecyloxy)hexyl]amino}octanoate (SM-102), respectively, for their COVID-19 mRNA vaccines, both of which have greater potency than MC3.^{[48,49]} The inventors considered if the PLA-b-PCB-X PLNPs would also increase the potency of these LNPs. We used our best-performing polymer (PLA-b-PCB-MOM) to produce 25/25 and 12.5/37.5 PLA-b-PCB-MOM/ionizable lipid (ALC-0315 or SM-102) PLNPs. Similar to the MC3 formulations, incorporating PLA-b-PCB-MOM into ALC-0315 and SM-102 increased FLuc knockdown compared to the ionizable lipid alone (FIG. 4C, FIG. 4D): all formulations containing PLA-b-PCB-MOM had similar IC₅₀ values of 0.2-0.3 nM, which were 3-fold and 5-fold less than conventional SM-102 and ALC-0315 formulations, respectively (FIG. 4E). As with the MC3 PLNPs, ALC-0315 and SM-102 PLNPs had no additional toxicity as measured by the PrestoBlue assay (FIG. 23B). Thus, incorporating our acid-responsive PLA-b-PCB-MOM in LNPs is efficacious and broadly applicable.

To validate the results disclosed herein, the inventors targeted an undruggable, clinically relevant gene, cyclin E1 (CCNE1), which is a cell cycle marker associated with tumour aggressiveness and drug resistance in breast cancer.^{[50-52]} The inventors tested our formulations with a palbociclib-resistant T-47D breast cancer cell line, which overexpressed CCNE1.^{[53]} The inventors chose to use formulations with SM-102 because they had the lowest IC₅₀ of the three ionizable lipids tested in our SK-OV-3 studies. The inventors encapsulated siRNA against CCNE1 (siCCNE1) in 12.5/37.5 PLA-b-PCB-MOM/SM-102 PLNPs vs. in SM-102 LNP formulations (and a negative control of 12.5/37.5 PLA-b-PCB-MOM/SM-102 encapsulating siFLuc). The inventors confirmed that our PLNPs were similarly cytocompatible compared to the SM-102 formulation, using the PrestoBlue assay as a proxy for cell viability (FIG. 4F). The expression of CCNE1 mRNA (measured by qRT-PCR) was significantly lower using the PLNP vs. SM-102 formulations (FIG. 4G). Therefore, siRNA delivery is enhanced with the inclusion of our acid-responsive polymer into LNPs and this is generalizable across multiple lipid mixtures, gene targets and cell types.

PLNPs Increase siRNA Bioavailability in the Cytosol

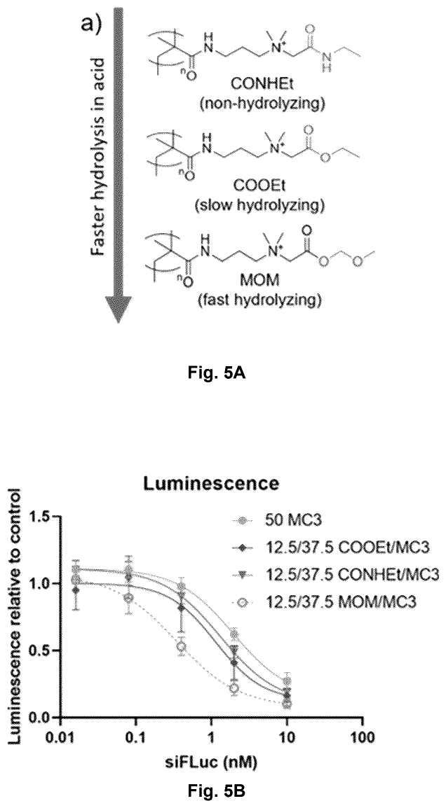

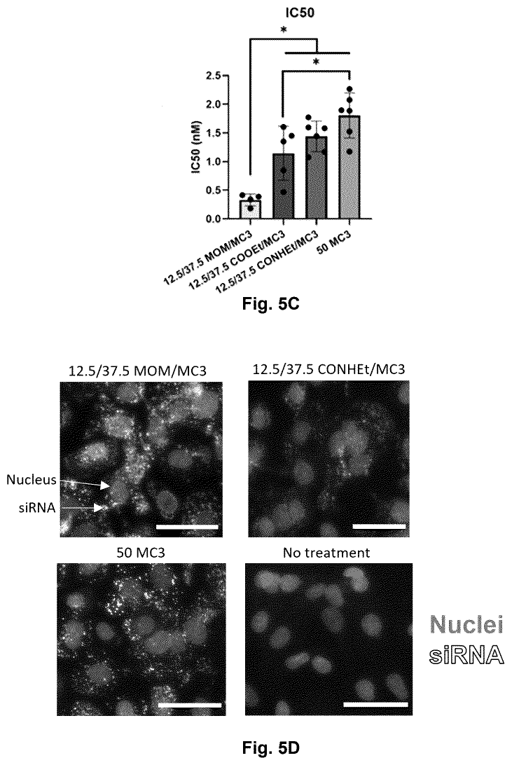

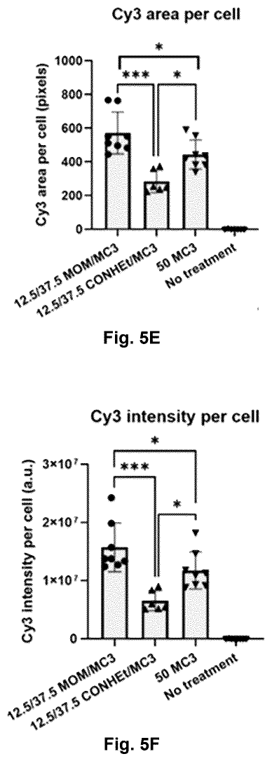

The inventors wondered whether the RNA-releasing properties of our polymers were responsible for the enhanced siRNA delivery. To answer this question, the inventors repeated the FLuc knockdown assay with two new PLNPs: (1) non-hydrolyzing 12.5/37.5 PLA-b-PCB-CONHEt/MC3 and (2) slow hydrolyzing 12.5/37.5 PLA-b-PCB-COOEt/MC3 (FIG. 5A). The inventors hypothesized that if the hydrolysis of the pendant group led to greater RNA release, then FLuc knockdown would be proportional to the rate of hydrolysis of the polymer. The inventors observed that PLA-b-PCB-CONHEt/MC3 PLNPs had similar potency to the conventional MC3 LNPs, and PLA-b-PCB-COOEt/MC3 PLNPs had an intermediate potency between that of PLA-b-PCB-MOM/MC3 PLNPs and the MC3 LNPs (FIG. 5B). PLA-b-PCB-MOM/MC3 was significantly more potent than all other formulations and PLA-b-PCB-COOEt/MC3 more potent than MC3 LNPs, as determined by lower IC₅₀ values (FIG. 5C). Thus, the acid-responsive hydrolysis of the hemiacetal groups in our polymers plays a significant role in the mechanism of RNA release from the PLNPs.

To visualize RNA in cells after internalization, the inventors delivered Cy3-labelled siRNA and used confocal microscopy to measure fluorescence (FIG. 5D). For all three NPs tested, the inventors observed both punctate and diffuse Cy3, likely indicating siRNA trapped in lysosomes and within the cytosol, respectively. The Cy3-siRNA signal observed with PLA-b-PCB-MOM/MC3 PLNPs covered significantly more area per cell than the MC3 LNPs (FIG. 5E), suggesting that more Cy3-siRNA escaped the endosome. This was further confirmed with Cy3 pixel intensity per cell (FIG. 5F), which was greatest when siRNA was delivered using the PLA-b-PCB-MOM/MC3 PLNPs, reflecting a higher concentration of RNA in the cell than when released from MC3 LNPs. Based on Cy3 area measurements, the inventors estimate that approximately 30% more siRNA is released into the cytosol by PLA-b-PCB-MOM/MC3 PLNPs than MC3 LNPs. The higher concentration observed in the cytosol is consistent with our proposed mechanism of RNA being released from the PLNP within the acidic endosome.

### Uptake and Endosomal Escape of PLNPs

The inventors examined whether the PLNPs had different uptake and endosomal escape than LNPs by confocal microscopy. To do this, the inventors encapsulated DiD in the PLNPs and delivered them to SK-OV-3 cells expressing an mCherry-galectin 9 fusion protein (mChG9). When endosomal disruption occurs, galectin 9 will accumulate on the interior of the disrupted endosome, which will be visible as mCherry puncti,^{[54]} as the inventors also observed here. This allowed us to detect PLNP uptake and endosomal escape simultaneously.

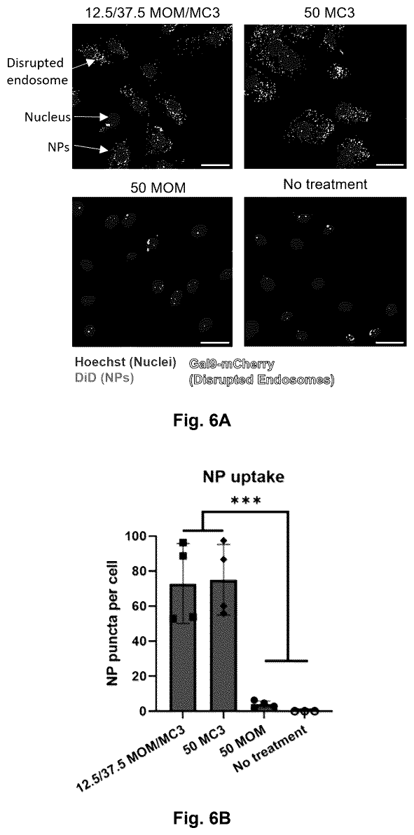

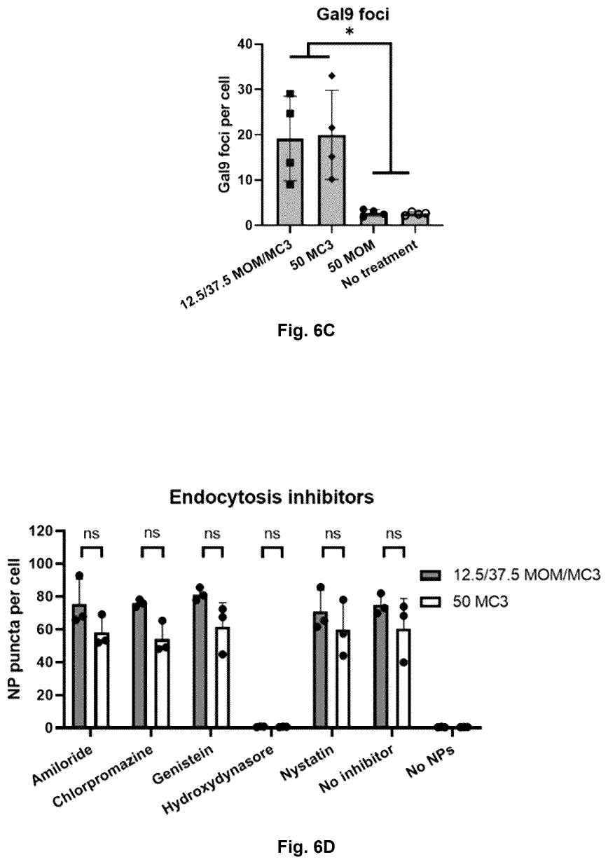

SK-OV-3 cells were treated with 25 nM of siFLuc delivered with either 12.5/37.5 PLA-b-PCB-MOM/MC3 PLNPs, 50/0 PLA-b-PCB-MOM/MC3 PLNPs or 50 MC3 LNPs (FIG. 6A). Strikingly, uptake was observed for both 12.5/37.5 PLA-b-PCB-MOM/MC3 and 50 MC3 LNPs, but not for the 50/0 PLA-b-PCB-MOM/MC3 PLNPs, indicating that MC3 is essential for uptake in our system (FIG. 6B). A similar trend was observed with endosomal disruption where 12.5/37.5 PLA-b-PCB-MOM/MC3 PLNPs and 50 MC3 LNPs both had elevated amounts of galectin 9 puncta (FIG. 6C). In both uptake and endosomal escape, there were no significant differences between the 12.5/37.5 PLA-b-PCB-MOM/MC3 PLNPs and the 50 MC3 LNPs and thus these factors do not explain the increased RNA delivery efficiency for the PLNPs compared to MC3 LNPs.

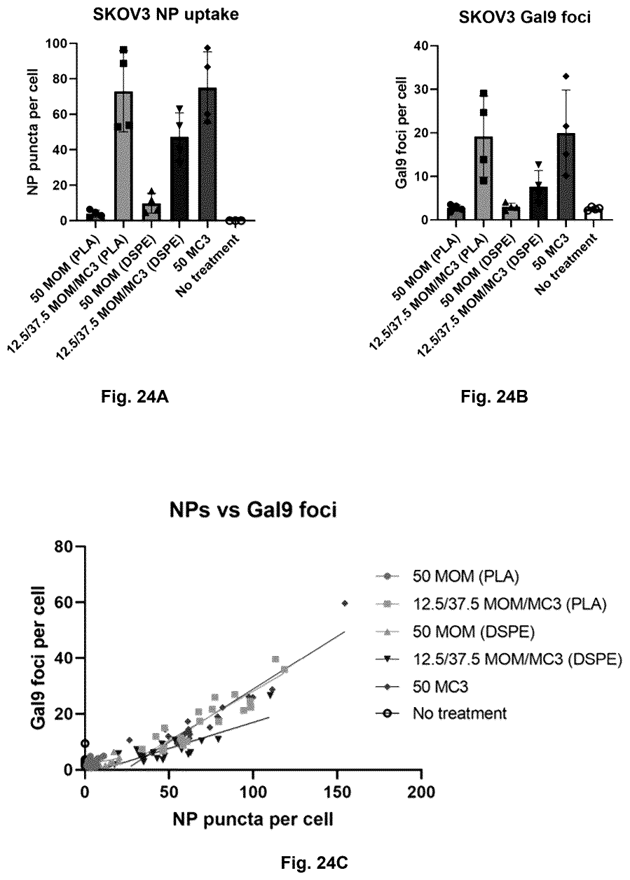

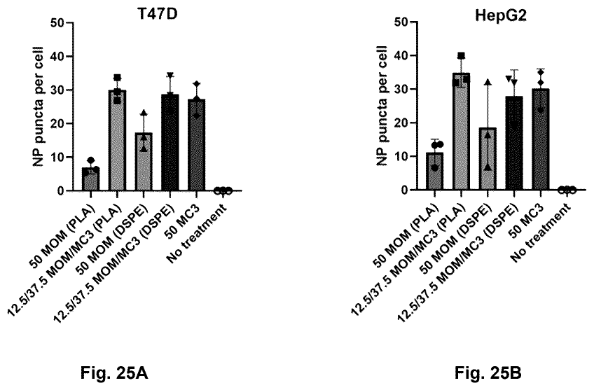

The inventors then wondered if the PLA hydrophobic block influenced uptake and endosomal escape, and thus the inventors repeated the experiments with DSPE-PCB-X PLNPs. Like 50/0 PLA-b-PCB-X/MC3 PLNPs, 50/0 DSPE-PCB-MOM/MC3 PLNPs were not taken up significantly, demonstrating the importance of having MC3 in the formulation for cell uptake (FIG. 24A). Interestingly, 12.5/37.5 PLA-b-PCB-MOM/MC3 resulted in higher uptake and greater endosomal disruption (as measured by Gal9 puncta) than that for 12.5/37.5 DSPE-PCB-MOM/MC3 (FIG. 24A-24B). When endosomal disruption is plotted against NP puncta, the DSPE-PCB-MOM/MC3 PLNPs have a lower slope, which shows that they are less efficient than PLA-b-PCB-X/MC3 at disrupting endosomes after uptake (FIG. 24C). Previous studies have shown that the low membrane fluidity of 18:0 saturated lipids such as DSPE have lower transfection efficiencies compared to more fluid membranes due to decreased fusion with the endosomal membrane.^{[55,56]} Therefore, the amorphous morphology of the PLA anchor may be more favourable for interaction with the endosomal membrane than the lamellar morphology of the DSPE anchor.

In addition to PLNP morphology, the inventors wondered whether other factors, such as cell type, were responsible for the observed differences in uptake between PLNPs. To answer this question, the inventors tested cell uptake with two additional cell lines: (1) T-47D breast cancer cells as used for our CCNE1 knockdown assay and (2) HepG2 liver cancer cells, which were chosen because of the liver's propensity to take up LNPs.^{[57]} The inventors hypothesized that because these cells are found in fattier tissues than SK-OV-3 cells, they may prefer a lipid hydrophobic anchor over a polyester hydrophobic one. The inventors noticed that while the best and worst performing PLNPs remained the same, less NPs in general were taken up by both cell lines (FIG. 25A-25B). This finding was unsurprising because different cell lines are known to uptake NPs with varying efficiencies.^{[58]} Unlike with the SK-OV-3 cells, 12.5/37.5 DSPE-PCB-MOM/MC3 PLNPs performed similarly to the 12.5/37.5 PLA-b-PCB-MOM/MC3 PLNPs and the 50 MC3 LNPs. Thus, replacing the polymeric PLA with a fatty-acid DSPE may have helped increase uptake in these cell types. Though the inventors did not further explore the DSPE-PCB-X PLNPs, this discovery may be useful for future iterations of this technology for delivery to these target tissues as it is well reported that changing the lipid composition of LNPs can greatly affect their biodistribution.^{[59,60]}

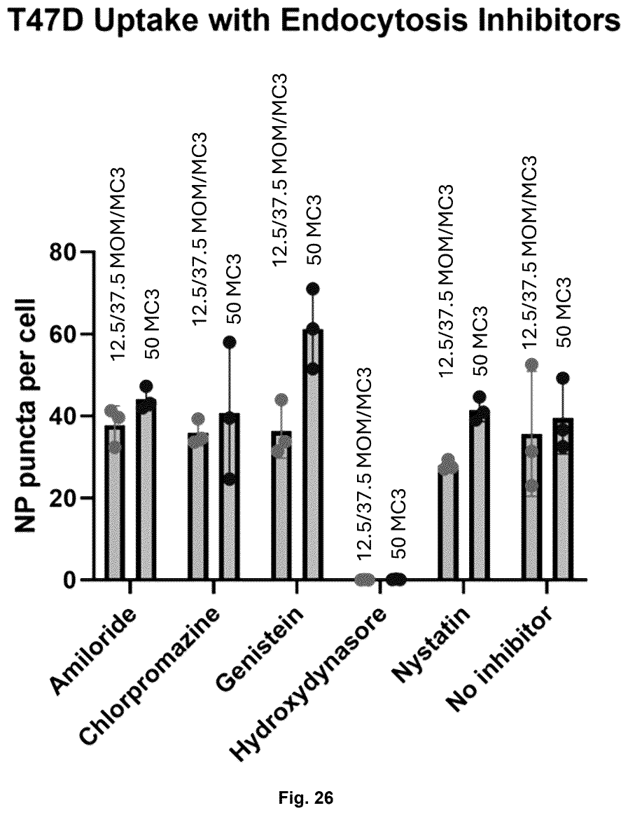

To further understand the uptake mechanism of PLA-b-PCB-X PLNPs, the inventors inhibited different endocytosis pathways using small molecules: amiloride (a macropinocytosis inhibitor), chlorpromazine (a clathrin-mediated endocytosis inhibitor), genistein (a caveolae-dependent and clathrin-independent endocytosis inhibitor), hydroxydynasore (a dynamin-mediated endocytosis inhibitor), and nystatin (a cholesterol-dependent endocytosis inhibitor).^{[61,62]} SK-OV-3 cells were pre-treated with one of each of the small molecule inhibitors and then dosed with either 12.5/37.5 PLA-b-PCB-MOM/MC3 PLNPs or 50 MC3 LNPs. Of all the inhibitors tested, only hydroxydynasore reduced (and in fact, abolished) uptake for both NPs (FIG. 6D). This result was also reproduced using T-47D cells, indicating that the uptake mechanism is consistent across different cell types (FIG. 26). Previous studies have shown that one of the most important proteins for internalization of LNPs is apolipoprotein E, which is recognized by the LDL receptor on the cell surface.^{[63,64]} Hydroxydynasore is likely an effective inhibitor of LNP uptake because endocytosis by LDL receptor is a dynamin-dependent process.^{[65]} The lack of inhibition by amiloride and chlorpromazine is somewhat surprising, as previous studies have demonstrated uptake in HeLa and Raw 264.7 cell lines by the mechanisms inhibited by these drugs.^{[3,61]} However, endocytic inhibition by small molecules has varying effectiveness across cell types,^{[66]} and thus it is possible that the SK-OV-3 cells used in our experiment are less sensitive to amiloride and chlorpromazine than previously tested cells. Notwithstanding these differences, the inventors demonstrated that our PLA-b-PCB-X PLNPs are taken up by the same mechanism as conventional LNPs.

PLNPs Enhance mRNA Delivery Compared to Conventional LNPs

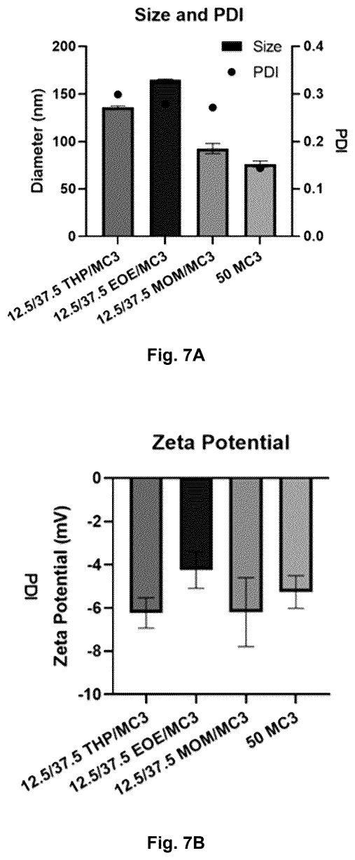

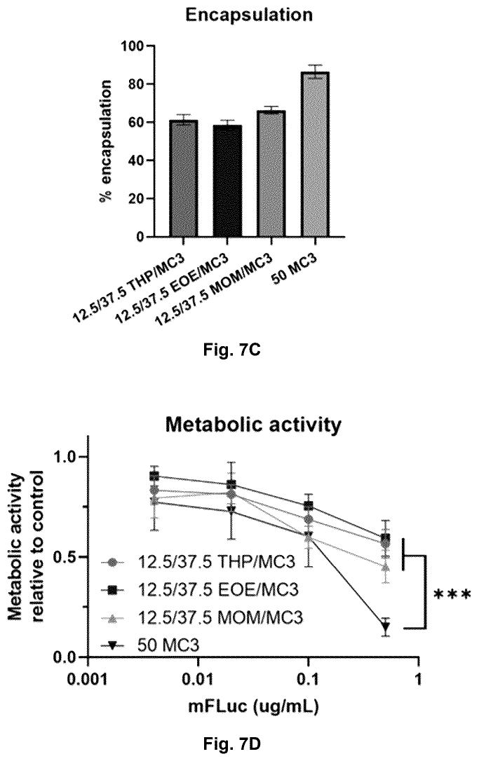

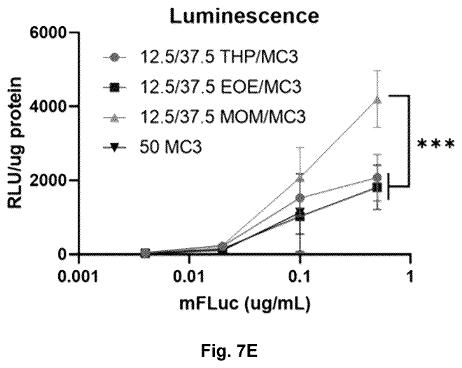

The inventors were curious whether our PLNPs would also be suitable for mRNA delivery. mRNA delivery is considerably more challenging than siRNA because it is larger and single-stranded, making it both more difficult to load into nanoparticles and more prone to degradation. The inventors switched our formulation method from microfluidic mixing (which had low mRNA encapsulation efficiency) to pipette mixing, and increased the N:P ratio from 6:1 to 10:1—a strategy that has been used by other groups to improve mRNA encapsulation in polymeric systems.^{[14,17,67]} These mRNA encapsulated PLNPs had similar physical characteristics to our siRNA PLNPs, with z-average diameters of 120-170 nm and PDIs of 0.2-0.3, as measured by DLS (FIG. 7A). However, the zeta potential was slightly more negative, ranging between −4 to −7 mV (FIG. 7B) in contrast to siRNA, which had slightly positive zeta potentials of +2 to +6 mV. By pipette mixing, the inventors achieved mRNA encapsulation efficiencies of 55-69% (FIG. 7C), which is admittedly lower than the gold standard (MC3 LNP at 88%), yet acceptable for testing in vitro.

The inventors delivered FLuc mRNA to SK-OV-3 cells using the PLNPs vs. MC3 LNPs alone and found that our 12.5/37.5 PLA-b-PCB-X/MC3 PLNPs retained significantly higher metabolic activity at the highest concentration tested of 0.5 μg/mL FLuc mRNA compared to conventional MC3 LNPs, (FIG. 7D). The inventors attribute the lower toxicity of our PLNPs to having a lower concentration of MC3, which can be toxic at high concentrations. Thus, the reduced toxicity of the PLNPs allowed us to deliver higher concentrations of mRNA, where the inventors observed the greatest luminescence, representing higher overall mRNA transfection (FIG. 7E). Of the three polymers tested, the inventors observed that PLA-b-PCB-MOM produced significantly more luminescence than the other two polymers without affecting cytotoxicity. The increased efficacy of the PLA-b-PCB-MOM polymer over the other two can be attributed to it having the smallest pendant group, which gives it the least steric hinderance to hydrolysis and least hydrophobic character. In contrast, the larger pendant groups of PLA-b-PCB-EOE and -THP may have more hydrophobic interactions with mRNA, which could both decrease hydrolysis by way of steric hinderance, and prevent full dissociation of mRNA after hydrolysis at acidic pH. Thus, our PLNPs (especially the PLA-b-PCB-MOM PLNPs) are advantageous for mRNA delivery due to both their RNA-releasing ability and their lower toxicity compared to conventional LNPs.

### CONCLUSIONS

The inventors have synthesized PLA-b-PCB-X polymers where X represents one of three acid-responsive hemiacetal pendant groups. These polymers were formulated with lipids into PLNPs that could encapsulate both siRNA and mRNA with high efficiency. PLNPs delivered RNA at higher efficiencies than conventional LNPs in a variety of cell lines. The inventors demonstrated the broad applicability of our approach with three different ionizable lipids, which is especially important as more formulations are optimized. The inventors determined that the PLNPs were internalized by the same mechanisms as conventional LNPs and induced endosomal escape to an equal degree. Moreover, the inventors found that acid-responsive release of RNA from the PLNPs was necessary for increased efficacy and confirmed that more siRNA was released to the cytosol using our PLNPs than traditional LNPs. Therefore, the inventors have demonstrated the importance of RNA dissociation from its nanocarrier after endocytosis and would emphasize this as a primary consideration when designing RNA nanocarriers.

In an embodiment there is provided a pH-sensitive polymer comprising:
a poly(lactic acid)-block-poly(carboxybetaine) derivative that is cationic and complexed with the nucleic acid at pH 7.4 and irreversibly converted to neutral and having a lower affinity to the nucleic acid at endosomal pH.

This poly(lactic acid)-block-poly(carboxybetaine) derivative is selected from the group consisting of amphiphilic, cationic block co-polymer PLA-b-PCB-THP, amphiphilic, cationic block co-polymer PLA-b-PCB-EOE and amphiphilic, cationic block co-polymer PLA-b-PCB-MOM. The poly(lactic acid)-block-poly(carboxybetaine) derivative is amphiphilic, cationic block co-polymer PLA-b-PCB-THP. Alternatively, the poly(lactic acid)-block-poly(carboxybetaine) derivative is amphiphilic, cationic block co-polymer PLA-b-PCB-EOE. Alternatively, the poly(lactic acid)-block-poly(carboxybetaine) derivative is amphiphilic, cationic block co-polymer PLA-b-PCB-MOM.

The present disclosure provides a method of synthesizing an amphiphilic, cationic block co-polymer PLA-b-PCB-THP, comprising:
synthesizing tetrahydro-2H-pyran-2-yl 2-bromoacetate (THP-Br); and
carrying out alkylation of PLA-b-PDMAPMA by THP-Br to produce amphiphilic, cationic block co-polymer PLA-b-PCB-THP.

The present disclosure also provides a method of synthesizing an amphiphilic, cationic block co-polymer PLA-b-PCB-EOE, comprising:
synthesizing 1-ethoxyethyl 2-bromoacetate (EOE-Br); and
carrying out alkylation of PLA-b-PDMAPMA by EOE-Br to produce amphiphilic, cationic block co-polymer PLA-b-PCB-EOE.

The present disclosure further provides a method of synthesizing an amphiphilic, cationic block co-polymer PLA-b-PCB-MOM, comprising:
synthesizing methoxymethyl 2-bromoacetate (MOM-Br); and
carrying out alkylation of PLA-b-PDMAPMA by MOM-Br to produce amphiphilic, cationic block co-polymer PLA-b-PCB-MOM.

The present disclosure provides a use of a poly(lactic acid)-block-poly(carboxybetaine) derivative for the preparation of lipid nanoparticles containing nucleic acids wherein the poly(lactic acid)-block-poly(carboxybetaine) derivative is cationic and complexed with the nucleic acids at pH 7.4 and irreversibly converted to neutral and having a lower affinity to the nucleic acids at endosomal pH.

In this use, the poly(lactic acid)-block-poly(carboxybetaine) derivative is selected from the group consisting of amphiphilic, cationic block co-polymer PLA-b-PCB-THP, amphiphilic, cationic block co-polymer PLA-b-PCB-EOE and amphiphilic, cationic block co-polymer PLA-b-PCB-MOM.

The poly(lactic acid)-block-poly(carboxybetaine) derivative is amphiphilic, cationic block co-polymer PLA-b-PCB-THP.

The poly(lactic acid)-block-poly(carboxybetaine) derivative is amphiphilic, cationic block co-polymer PLA-b-PCB-EOE.

The poly(lactic acid)-block-poly(carboxybetaine) derivative is amphiphilic, cationic block co-polymer PLA-b-PCB-MOM.

While the present invention has been described with reference to specific embodiments for illustrative purposes, it is not intended that the invention be limited to such embodiments, as the embodiments described herein are intended to be examples. It is to be understood that various alternatives, modifications, and equivalents of the embodiments described herein fall within the true spirit and scope of the invention, which should be given the broadest interpretation consistent with the application as a whole.

### REFERENCES

[1] G. M. Traber, A.-M. Yu, Mol Pharmacol 2024, 106, 13.
[2] H. Zogg, R. Singh, S. Ro, Int J Mol Sci 2022, 23, 2736.
[3] J. Gilleron, W. Querbes, A. Zeigerer, A. Borodovsky, G. Marsico, U. Schubert, K. Manygoats, S. Seifert, C. Andree, M. Stöter, H. Epstein-Barash, L. Zhang, V. Koteliansky, K. Fitzgerald, E. Fava, M. Bickle, Y. Kalaidzidis, A. Akinc, M. Maier, M. Zerial, Nat Biotechnol 2013, 31, 638.
[4] A. Wittrup, A. Ai, X. Liu, P. Hamar, R. Trifonova, K. Charisse, M. Manoharan, T. Kirchhausen, J. Lieberman, Nat Biotechnol 2015, 33, 870.
[5] P. Paramasivam, C. Franke, M. Stöter, A. Höijer, S. Bartesaghi, A. Sabirsh, L. Lindfors, M. Yanez Arteta, A. Dahlén, A. Bak, S. Andersson, Y. Kalaidzidis, M. Bickle, M. Zerial, Journal of Cell Biology 2022, 221, 1.
[6] Y. Lee, M. Jeong, J. Park, H. Jung, H. Lee, Immunogenicity of lipid nanoparticles and its impact on the efficacy of mRNA vaccines and therapeutics, Vol. 55, Springer Nature, 2023, pp. 2085-2096.
[7] S. Ndeupen, Z. Qin, S. Jacobsen, A. Bouteau, H. Estanbouli, B. Z. Igyártó, iScience 2021, 24.
[8] R. Kedmi, N. Ben-Arie, D. Peer, Biomaterials 2010, 31, 6867.
[9] A. Biscans, S. Ly, N. McHugh, D. A. Cooper, A. Khvorova, Journal of Controlled Release 2022, 349, 831.
[10] A. Ahmad, J. M. Khan, S. Haque, Strategies in the design of endosomolytic agents for facilitating endosomal escape in nanoparticles, Vol. 160, Elsevier B. V., 2019, pp. 61-75.
[11] B. Yu, X. Wang, C. Zhou, L. Teng, W. Ren, Z. Yang, C.-H. Shih, T. Wang, R. J. Lee, S. Tang, L. J. Lee, Pharm Res 2014, 31, 2685.
[12] G. Sahay, W. Querbes, C. Alabi, A. Eltoukhy, S. Sarkar, C. Zurenko, E. Karagiannis, K. Love, D. Chen, R. Zoncu, Y. Buganim, A. Schroeder, R. Langer, D. G. Anderson, Nat Biotechnol 2013, 31, 653.
[13] C. T. Greco, T. H. Epps, M. O. Sullivan, ACS Biomater Sci Eng 2016, 2, 1582.
[14] C. J. McKinlay, J. R. Vargas, T. R. Blake, J. W. Hardy, M. Kanada, C. H. Contag, P. A. Wender, R. M. Waymouth, Proceedings of the National Academy of Sciences 2017, 114, E448.
[15] S. A. Smith, B. Rossi Herling, C. Zhang, M. A. Beach, S. L. Y. Teo, E. R. Gillies, A. P. R. Johnston, G. K. Such, Biomacromolecules 2023, 24, 4958.
[16] Q. E. A. Sirianni, T. Wang, A. Borecki, Z. Deng, J. A. Ronald, E. R. Gillies, Biomater Sci 2022, 10, 2557.
[17] C. J. McKinlay, N. L. Benner, O. A. Haabeth, R. M. Waymouth, P. A. Wender, Proceedings of the National Academy of Sciences 2018, 115, E5859.
[18] S. M. Moghimi, P. Symonds, J. C. Murray, A. C. Hunter, G. Debska, A. Szewczyk, Molecular Therapy 2005, 11, 990.
[19] X. R. Shao, X. Q. Wei, X. Song, L. Y. Hao, X. X. Cai, Z. R. Zhang, Q. Peng, Y. F. Lin, Cell Prolif 2015, 48, 465.
[20] C. Tros de llarduya, Y. Sun, N. Düzgüneş, Gene delivery by lipoplexes and polyplexes, Vol. 40, 2010, pp. 159-170.
[21] W. Cai, T. Luo, X. Chen, L. Mao, M. Wang, Adv Funct Mater 2022, 32.
[22] J. J. Røise, H. Han, J. Li, D. L. Kerr, C. Taing, K. Behrouzi, M. He, E. Ruan, L. Y. Chan, E. M. Espinoza, S. Reinhard, K. Thakker, J. Kwon, M. R. K. Mofrad, N. Murthy, Mol Pharm 2022, 19, 67.
[23] B. Li, I. O. Raji, A. G. R. Gordon, L. Sun, T. M. Raimondo, F. A. Oladimeji, A. Y. Jiang, A. Varley, R. S. Langer, D. G. Anderson, Nat Mater 2024.
[24] L. Xue, A. G. Hamilton, G. Zhao, Z. Xiao, R. El-Mayta, X. Han, N. Gong, X. Xiong, J. Xu, C. G. Figueroa-Espada, S. J. Shepherd, A. J. Mukalel, M. G. Alameh, J. Cui, K. Wang, A. E. Vaughan, D. Weissman, M. J. Mitchell, Nat Commun 2024, 15.
[25] K. A. Hajj, R. L. Ball, S. B. Deluty, S. R. Singh, D. Strelkova, C. M. Knapp, K. A. Whitehead, Small 2019, 15, 1.
[26] A. Aljabbari, A. G. Lokras, J. J. K. Kirkensgaard, T. Rades, H. Franzyk, A. Thakur, Y. Zhang, C. Foged, J Colloid Interface Sci 2023, 633, 907.
[27] K. Thanki, X. Zeng, S. Justesen, S. Tejlmann, E. Falkenberg, E. Van Driessche, H. Mørck Nielsen, H. Franzyk, C. Foged, European Journal of Pharmaceutics and Biopharmaceutics 2017, 120, 22.
[28] S. Colombo, D. Cun, K. Remaut, M. Bunker, J. Zhang, B. Martin-Bertelsen, A. Yaghmur, K. Braeckmans, H. M. Nielsen, C. Foged, Journal of Controlled Release 2015, 201, 22.
[29] J. C. Kaczmarek, K. J. Kauffman, O. S. Fenton, K. Sadtler, A. K. Patel, M. W. Heartlein, F. DeRosa, D. G. Anderson, Nano Lett 2018, 18, 6449.
[30] J. C. Kaczmarek, A. K. Patel, K. J. Kauffman, O. S. Fenton, M. J. Webber, M. W. Heartlein, F. DeRosa, D. G. Anderson, Angewandte Chemie—International Edition 2016, 55, 13808.
[31] X. Su, J. Fricke, D. G. Kavanagh, D. J. Irvine, Mol Pharm 2011, 8, 774.
[32] C. D. Siewert, H. Haas, V. Cornet, S. S. Nogueira, T. Nawroth, L. Uebbing, A. Ziller, J. Al-Gousous, A. Radulescu, M. A. Schroer, C. E. Blanchet, D. I. Svergun, M. P. Radsak, U. Sahin, P. Langguth, Cells 2020, 9, 1.
[33] T. H. Cheung, C. Xue, D. A. Kurtz, M. S. Shoichet, ACS Appl Mater Interfaces 2023, 15, 50560.
[34] A. Sharma, I. Ramos-Tomillero, A. El-Faham, E. Nicolas, H. Rodriguez, B. G. de la Torre, F. Albericio, Understanding Tetrahydropyranyl as a Protecting Group in Peptide Chemistry, Vol. 6, Wiley-VCH Verlag, 2017, pp. 168-177.
[35] Z. Cao, Q. Yu, H. Xue, G. Cheng, S. Jiang, Angewandte Chemie International Edition 2010, 49, 3771.
[36] K. P. Garcia, K. Zarschler, L. Barbaro, J. A. Barreto, W. O'Malley, L. Spiccia, H. Stephan, B. Graham, Zwitterionic-coated “stealth” nanoparticles for biomedical applications: Recent advances in countering biomolecular corona formation and uptake by the mononuclear phagocyte system, Vol. 10, Wiley-VCH Verlag, 2014, pp. 2516-2529.
[37] N. Oh, J. H. Park, Endocytosis and exocytosis of nanoparticles in mammalian cells, Vol. 9, Dove Medical Press Ltd., 2014, pp. 51-63.
[38] M. Jayaraman, S. M. Ansell, B. L. Mui, Y. K. Tam, J. Chen, X. Du, D. Butler, L. Eltepu, S. Matsuda, J. K. Narayanannair, K. G. Rajeev, I. M. Hafez, A. Akinc, M. A. Maier, M. A. Tracy, P. R. Cullis, T. D. Madden, M. Manoharan, M. J. Hope, Angewandte Chemie—International Edition 2012, 51, 8529.
[39] C. Walsh, K. Ou, N. M. Belliveau, T. J. Leaver, A. W. Wild, J. Huft, P. J. Lin, S. Chen, A. K. Leung, J. B. Lee, C. L. Hansen, R. J. Taylor, E. C. Ramsay, P. R. Cullis, Methods in Molecular Biology 2014, 1141, 109.
[40] M. Maeki, S. Uno, A. Niwa, Y. Okada, M. Tokeshi, Journal of Controlled Release 2022, 344, 80.
[41] Y. B. Hu, E. B. Dammer, R. J. Ren, G. Wang, Transl Neurodegener 2015, 4, 1.
[42] J. Wang, S. Yuan, Y. Zhang, W. Wu, Y. Hu, X. Jiang, Biomater Sci 2016, 4, 1351.
[43] Y. Schilt, T. Berman, X. Wei, Y. Barenholz, U. Raviv, Biochim Biophys Acta Gen Subj 2016, 1860, 108.
[44] R. Nordström, L. Zhu, J. Härmark, Y. Levi-Kalisman, E. Koren, Y. Barenholz, G. Levinton, D. Shamrakov, Pharmaceutics 2021, 13, 1.
[45] M. H. Y. Cheng, J. Leung, Y. Zhang, C. Strong, G. Basha, A. Momeni, Y. Chen, E. Jan, A. Abdolahzadeh, X. Wang, J. A. Kulkarni, D. Witzigmann, P. R. Cullis, Advanced Materials 2023, 2303370.
[46] J. A. Kulkarni, M. M. Darjuan, J. E. Mercer, S. Chen, R. van der Meel, J. L. Thewalt, Y. Y. C. Tam, P. R. Cullis, ACS Nano 2018, 12, 4787.
[47] Z. Cao, L. Zhang, S. Jiang, Langmuir 2012, 28, 11625.
[48] F. Ferraresso, A. W. Strilchuk, L. J. Juang, L. G. Poole, J. P. Luyendyk, C. J. Kastrup, Mol Pharm 2022, 19, 2175.
[49] K. J. Hassett, K. E. Benenato, E. Jacquinet, A. Lee, A. Woods, O. Yuzhakov, S. Himansu, J. Deterling, B. M. Geilich, T. Ketova, C. Mihai, A. Lynn, I. McFadyen, M. J. Moore, J. J. Senn, M. G. Stanton, O. Almarsson, G. Ciaramella, L. A. Brito, Mol Ther Nucleic Acids 2019, 15, 1.
[50] H. H. Milioli, S. Alexandrou, E. Lim, C. E. Caldon, Cyclin E1 and cyclin E2 in ER+breast cancer: Prospects as biomarkers and therapeutic targets, Vol. 27, BioScientifica Ltd., 2020, pp. R93-R112.
[51] C. E. Caldon, C. M. Sergio, J. Kang, A. Muthukaruppan, M. N. Boersma, A. Stone, J. Barraclough, C. S. Lee, M. A. Black, L. D. Miller, J. M. Gee, R. I. Nicholson, R. L. Sutherland, C. G. Print, E. A. Musgrove, Mol Cancer Ther 2012, 11, 1488.
[52] Z. M. Zhao, S. E. Yost, K. E. Hutchinson, S. M. Li, Y. C. Yuan, J. Noorbakhsh, Z. Liu, C. Warden, R. M. Johnson, X. Wu, J. H. Chuang, Y. Yuan, BMC Cancer 2019, 19.
[53] I. Soria-Bretones, K. L. Thu, J. Silvester, J. Cruickshank, S. El Ghamrasni, W. Ba-alawi, G. C. Fletcher, R. Kiarash, M. J. Elliott, J. J. Chalmers, A. C. Elia, A. Cheng, A. A. N Rose, M. R. Bray, B. Haibe-Kains, T. W. Mak, D. W. Cescon, The spindle assembly checkpoint is a therapeutic vulnerability of CDK4/6 inhibitor-resistant ER+breast cancer with mitotic aberrations, Vol. 8, 2022.
[54] M. J. Munson, G. O'Driscoll, A. M. Silva, E. Lázaro-Ibáñez, A. Gallud, J. T. Wilson, A. Collén, E. K. Esbjörner, A. Sabirsh, Commun Biol 2021, 4.
[55] E. Yuba, Y. Nakajima, K. Tsukamoto, S. Iwashita, C. Kojima, A. Harada, K. Kono, Journal of Controlled Release 2012, 160, 552.
[56] H. Abumanhal-Masarweh, D. da Silva, M. Poley, A. Zinger, E. Goldman, N. Krinsky, R. Kleiner, G. Shenbach, J. E. Schroeder, J. Shklover, J. Shainsky-Roitman, A. Schroeder, Journal of Controlled Release 2019, 307, 331.
[57] W. Ngo, S. Ahmed, C. Blackadar, B. Bussin, Q. Ji, S. M. Mladjenovic, Z. Sepahi, W. C. W. Chan, Adv Drug Deliv Rev 2022, 185, 114238.
[58] T. Dos Santos, J. Varela, I. Lynch, A. Salvati, K. A. Dawson, Small 2011, 7, 3341.
[59] R. Pattipeiluhu, G. Arias-Alpizar, G. Basha, K. Y. T. Chan, J. Bussmann, T. H. Sharp, M. Moradi, N. Sommerdijk, E. N. Harris, P. R. Cullis, A. Kros, D. Witzigmann, F. Campbell, Advanced Materials 2022, U.S. Pat. Nos. 2,201,095, 2,201,095.
[60] A. Idris, A. Davis, A. Supramaniam, D. Acharya, G. Kelly, Y. Tayyar, N. West, P. Zhang, C. L. D. McMillan, C. Soemardy, R. Ray, D. O'Meally, T. A. Scott, N. A. J. McMillan, K. V. Morris, Molecular Therapy 2021, 29, 1.
[61] P. J. C. Lin, Y. Y. C. Tam, I. Hafez, A. Sandhu, S. Chen, M. A. Ciufolini, I. R. Nabi, P. R. Cullis, Nanomedicine 2013, 9, 233.
[62] E. N. Donders, K. V. Slaughter, C. Dank, A. N. Ganesh, B. K. Shoichet, M. Lautens, M. S. Shoichet, Advanced Science 2023, 2300311.
[63] F. Sebastiani, M. Yanez Arteta, M. Lerche, L. Porcar, C. Lang, R. A. Bragg, C. S. Elmore, V. R. Krishnamurthy, R. A. Russell, T. Darwish, H. Pichler, S. Waldie, M. Moulin, M. Haertlein, V. T. Forsyth, L. Lindfors, M. Cárdenas, ACS Nano 2021, 15, 6709.
[64] V. Francia, R. M. Schiffelers, P. R. Cullis, D. Witzigmann, Bioconjug Chem 2020, 31, 2046.
[65] K. L. Prichard, N. S. O'Brien, S. R. Murcia, J. R. Baker, A. McCluskey, Front Cell Neurosci 2022, 15.
[66] D. Vercauteren, R. E. Vandenbroucke, A. T. Jones, J. Rejman, J. Demeester, S. C. De Smedt, N. N. Sanders, K. Braeckmans, Molecular Therapy 2010, 18, 561.
[67] S. Liu, X. Wang, X. Yu, Q. Cheng, L. T. Johnson, S. Chatterjee, D. Zhang, S. M. Lee, Y. Sun, T.-C. Lin, J. L. Liu, D. J. Siegwart, J Am Chem Soc 2021, 143, 21321.
[68] K. V. Kilchrist, S. C. Dimobi, M. A. Jackson, B. C. Evans, T. A. Werfel, E. A. Dailing, S. K. Bedingfield, I. B. Kelly, C. L. Duvall, ACS Nano 2019, 13, 1136.
[1] T. H. Cheung, C. Xue, D. A. Kurtz, M. S. Shoichet, ACS Appl Mater Interfaces 2023, 15, 50560.

Therefore what is claimed is:

1. A pH-sensitive polymer comprising:
a poly(lactic acid)-block-poly(carboxybetaine) derivative that is cationic and complexed with a nucleic acid at pH 7.4 and irreversibly converted to neutral and having a lower affinity to the nucleic acid at endosomal pH.

2. The pH-sensitive polymer according to claim 1, wherein the poly(lactic acid)-block-poly(carboxybetaine) derivative is selected from the group consisting of amphiphilic, cationic block co-polymer PLA-b-PCB-THP; amphiphilic, cationic block co-polymer PLA-b-PCB-EOE; and amphiphilic, cationic block co-polymer PLA-b-PCB-MOM.

3. The pH-sensitive polymer according to claim 2, wherein the poly(lactic acid)-block-poly(carboxybetaine) derivative is amphiphilic, cationic block co-polymer PLA-b-PCB-THP.

4. The pH-sensitive polymer according to claim 2, wherein the poly(lactic acid)-block-poly(carboxybetaine) derivative is amphiphilic, cationic block co-polymer PLA-b-PCB-EOE.

5. The pH-sensitive polymer according to claim 2, wherein the poly(lactic acid)-block-poly(carboxybetaine) derivative is amphiphilic, cationic block co-polymer PLA-b-PCB-MOM.

6. A method of synthesizing a pH-sensitive cationic block co-polymer comprising a poly(lactic acid)-block-poly(carboxybetaine) derivative of formula: PLA-b-PCB-X, the method comprising the steps of
synthesizing an intermediate of formula X-Br; and
carrying out alkylation of PLA-b-PDMAPMA by X-Br to produce amphiphilic, cationic block co-polymer PLA-b-PCB-X,
wherein X-Br is tetrahydro-2H-pyran-2-yl 2-bromoacetate (THP-Br), 1-ethoxyethyl 2-bromoacetate (EOE-Br) or methoxymethyl 2-bromoacetate (MOM-Br) and the block co-polymer PLA-b-PCB-X is PLA-b-PCB-THP, PLA-b-PCB-EOE or PLA-b-PCB-MOM.

7. The method of claim 6, wherein PLA-b-PCB-X is PLA-b-PCB-THP and X-Br is tetrahydro-2H-pyran-2-yl 2-bromoacetate (THP-Br).

8. The method of claim 6, wherein PLA-b-PCB-X is PLA-b-PCB-EOE and X-Br is 1-ethoxyethyl 2-bromoacetate (EOE-Br).

9. The method of claim 6, wherein PLA-b-PCB-X is PLA-b-PCB-MOM and X-Br is methoxymethyl 2-bromoacetate (MOM-Br).

10. Use of the poly(lactic acid)-block-poly(carboxybetaine) derivative of claim 1 for preparation of lipid nanoparticles containing nucleic acids.

11. The use of claim 10, wherein the nucleic acids are RNA.

12. The use of claim 11, wherein the RNA is mRNA or siRNA.

13. A method of delivering RNA to cytosol of cells, using the pH-sensitive polymer of claim 1, the method comprising:
(a) preparing a formulation of lipid nanoparticles with RNA in the presence of the pH-sensitive polymer, and
(b) transfecting cells with the formulation obtained from step (a).

14. The method of claim 13, wherein the RNA is mRNA or siRNA.

15. A pH-sensitive polymer comprising:
a poly(lactic acid)-block-poly(carboxybetaine) derivative having pendant carboxylate groups protected with hemiacetal esters.

16. The pH-sensitive polymer of claim 15, wherein the poly(lactic acid)-block-poly(carboxybetaine) derivative is selected from the group consisting of amphiphilic, cationic block co-polymer PLA-b-PCB-THP; amphiphilic, cationic block co-polymer PLA-b-PCB-EOE; and amphiphilic, cationic block co-polymer PLA-b-PCB-MOM.

17. Use of the poly(lactic acid)-block-poly(carboxybetaine) derivative of claim 16 for the preparation of lipid nanoparticles containing nucleic acids.

18. The use of claim 17, wherein the nucleic acids are RNA.

19. A method of delivering RNA to cytosol of cells, using the pH-sensitive polymer of claim 15, the method comprising:
(a) preparing a formulation of lipid nanoparticles with RNA in the presence of the pH-sensitive polymer; and
(b) transfecting cells with the formulation obtained from step (a).

20. The method of claim 19, wherein the RNA is mRNA or siRNA.
